SEQUENCE LISTING (40 sequences)

SEQ ID NO: 1 - protein, 1230 aa, Homo sapiens [sequence omitted]

SEQ ID NO: 2 - DNA, 3832 bp, Homo sapiens
cggcggcggcggcgcctggagcggcgtgcgtaatgtcggcactttgctggggccgcggagcggcggggctcaaacgagccctacggccttgcggccgcccgggactccccggcaaagaagggaccgccggtggcgtctgcggccccaggaggagctcgtccgccagcccccaggagcaagaccaagaccgcaggaaggactggggccacgtggagctgctggaggtgctccaggcgcgggtgcggcagctgcaggctgagagcgtgtcggaggtggtggtgaacagggtggatgtggcgcggctcccagaatgtggcagtggagatggtagcctccagccacccaggaaggtccagatgggggccaaggatgccaccccggtgccctgtggccgctgggcaaagatactggagaaggataagcggacccagcagatgcgtatgcagcggttgaaggcgaagctgcagatgccattccagagcggggagttcaaggcgctgaccaggcgcctgcaggtggagccccggctcctgagcaagcagatggccgggtgcctggaggactgcacgcgccaggcccccgagagcccctgggaggagcagctggcccggctgctgcaggaggcccctgggaagctgagcctcgatgtggagcaggccccgtcggggcagcactcgcaggcccagctctcaggtcagcagcagaggctcctggccttcttcaagtgctgcctgctcactgaccagctgcccctcgcccaccacctgctggtcgtccaccacggccagcggcagaagcggaagctgctcacgctggacatgtacaacgccgtgatgcttggctgggcgcggcagggtgctttcaaggagctggtatatgtgttattcatggtgaaggatgccggcttgaccccggacctgctgtcctatgcggctgccctccagtgcatggggaggcaggaccaggacgccgggaccatcgaaaggtgtctggaacagatgagccaggaggggctgaagctgcaggcactcttcaccgccgttctgctgtctgaggaggatcgggccactgttctgaaggccgtgcacaaggtgaagcccaccttcagcctcccgccgcagctgccgcccccggtcaacacctccaagctgctcagggacgtgtatgccaaggatgggcgtgtgtcctacccgaagctgcacctgcccttgaagaccctgcagtgcttctttgagaagcagctccacatggagctggccagcagggtgtgcgtggtgtccgtggagaagcccacgttgccaagcaaggaggtcaagcacgcgcggaagaccctgaagaccctgcgggaccaatgggagaaagcactgtgccgggcgctgcgggagaccaagaaccgcctagagcgcgaggtgtacgagggccggttctcactttaccccttcctgtgcctgctggacgagcgcgaggtggtgcggatgctcctgcaggtcctgcaggcgctgcccgcccaaggtgagtccttcaccaccctggcccgggagctgagtgcgcgcactttcagccggcacgtggtgcagaggcagcgggtcagtggccaggtgcaggcgctgcagaaccactacaggaagtacctctgcttgctggcctccgacgccgaggtgcccgagccctgcctgccgcggcagtactgggaggagctgggggcgcccgaggccctgcgggagcagccctggcccctgccagtgcagatggagctgggcaagctgctggcggagatgctggtgcaggctacgcagatgccatgcagcctggacaagccgcatcgttcctctcggcttgtccccgtgctctaccacgtgtattccttccgcaacgtccagcaaatcggcatcctgaagccgcacccggcctacgtgcagctgctggagaaggccgcggaacccacgctgaccttcgaggcggtggatgtacccatgctttgccccccgctgccctggacatcgccgcactctggtgctttcctgctcagccccaccaagctgatgcgcacggtggaaggcgccacgcaacaccaggagctgctggaaacctgcccacccaccgcgctgcatggcgcactggacgccctcacccaactgggcaactgcgcctggcgcgtcaacgggcgcgtgctggacctggtgctgcagctcttccaggccaagggctgcccccagctaggcgtgccggccccgccctccgaggcgccccagccgcccgaggcccacctgccgcacagcgccgcgcccgcccgcaaggccgagctgcgccgtgagctggcgcactgccagaaggtggcccgggagatgcacagcctgcgggcggaggcgctgtaccgcctctcgctggcgcagcacctgcgggaccgcgtcttctggctgccgcacaacatggacttccgcggccgcacctacccctgcccgccgcacttcaaccacctgggcagcgacgtggcgcgggccctgctggagttcgcccagggccgcccgctcggcccgcacggcctggattggctcaagatccacctggtcaatctcacggggttgaagaagcgggagccgctgcggaagcgcctggcctttgcggaggaggtgatggatgacatcctggactccgcggaccaacccttgacgggccgaaagtggtggatgggcgcggaggaaccctggcagacgctggcctgctgtatggaggtggcgaacgctgtgcgcgcctccgaccctgccgcctatgtctcccacctccccgtccatcaggacggctcttgcaacggcctgcagcattatgctgctctgggccgcgacagcgtgggcgccgcctccgtcaacctggagccctcggatgtgccgcaggacgtgtacagcggcgtggccgcgcaggtggaggtgttccgtaggcaggacgcccagcggggcatgcgggtggcacaggtgctggaaagtttcatcacccgcaaggtggtgaagcagacggtgatgacggtggtgtacggggtcacgcgctatggcgggcgcctgcagattgagaagcgcctccgggagctgagcgactttccccaggagttcgtgtgggaggcctctcactatctcgtacgccaggtcttcaagagtctacaggagatgttctcggggacccgggccatccagcactggctgaccgagagtgcccgcctcatctcccacatgggctctgtggtggagtgggtcacacccctgggcgtccccgtcatccagccgtatcgcctggactccaaggtcaagcaaataggaggtggaattcagagcatcacctacacccacaacggagacatcagccgaaagcccaacacacgtaagcagaagaacggcttcccgcccaacttcatccactcgctggactcctcccacatgatgctcaccgccctgcactgctacaggaagggcctgaccttcgtctctgtgcacgactgttactggactcacgcagctgatgtctccgtcatgaaccaggtgtgccgggagcagtttgtccgcttgcacagcgagcccatcctgcaggacctgtccagattcctggtcaagcggttctgctctgagccccagaagatcttggaggccagccagctgaaggagacactgcaggcggtgcccaagccaggggccttcgacctggagcaggtgaagcgttccacctacttcttcagctgacaccccgtgagccttgtcagtgtgtaaataaagctcttttgccaccccccaaaaaaaaaaaaaaaaaaaaaaaaaaaaaaaaaaaaaaaaaaaaaaaaaaaaaaaaa

SEQ ID NO: 3 - protein, 34 aa, Homo sapiens
MAFLRSMWGVLTALGRSGAELCTGCGSRLRSPFR

SEQ ID NO: 4 - DNA, 105 bp, Homo sapiens
atggcgtttctccgaagcatgtggggcgtgctgactgccctgggaaggtctggagcagagctgtgcaccggctgtggaagtcgactgcgctcccccttcaggtag

SEQ ID NO: 5 - protein, 346 aa, Homo sapiens
MAASGKLSTCRLPPLPTIREIIKLLRLQAANELSQNFLLDLRLTDKIVRKAGNLTNAYVYEVGPGPGGITRSILNADVAELLVVEKDTRFIPGLQMLSDAAPGKLRIVHGDVLTFKVEKAFSESLKRPWEDDPPNVHIIGNLPFSVSTPLIIKWLENISCRDGPFVYGRTQMTLTFQKEVAERLAANTGSKQRSRLSVMAQYLCNVRHIFTIPGQAFVPKPEVDVGVVHFTPLIQPKIEQPFKLVEKVVQNVFQFRRKYCHRGLRMLFPEAQRLESTGRLLELADIDPTLRPRQLSISHFKSLCDVYRKMCDEDPQLFAYNFREELKRRKSKNEEKEEDDAENYRL

SEQ ID NO: 6 - DNA, 1290 bp, Homo sapiens
gttacaatctgaagtgggagcggcggtgaaggtcctgggtgaggtaggggtggatggtgcttgccgcgtatcatggctgcctccggaaaactcagcacttgccgtctccctccgttgcccacgattcgagaaatcattaagttgttaagactgcaagcagcgaacgagctatcacagaatttcctcctggacttgaggctgacagataagattgtaaggaaagctggcaatctgacaaatgcttatgtttacgaagtgggccctgggccagggggaatcacaagatctattcttaatgccgacgtcgctgaacttctggtggttgaaaaggacactcgatttattcctggattacagatgctttctgatgcagcacctgggaaactgagaattgttcatggagatgtcttgacatttaaggtagaaaaggctttttcagaaagtcttaaaagaccctgggaagatgatcctccaaatgtacatattattggaaatctgccttttagtgtttcaactccactgattatcaagtggcttgaaaatatttcctgtagagatggaccttttgtttatggcagaactcagatgactttgacttttcaaaaggaagtggcagagagacttgcagccaatacaggaagcaaacagcgtagtcgcctctctgttatggctcagtacctctgcaatgttcgacacatctttacaattccaggacaagcttttgtccccaaaccagaggtggacgtgggcgtggtgcacttcactcccttgatacagcccaagatagagcagccattcaagctggtggaaaaagtggttcagaatgtatttcagttccgaaggaaatactgccatcgagggctcagaatgttattccctgaagcgcagcgcttggaaagcacgggcaggctgttagagttggcagacatagaccctactcttcggccccgccagctctccatctcacactttaagagcctctgtgatgtatacagaaaaatgtgtgatgaagacccacaactctttgcatataatttcagagaagaactcaagcgaagaaaaagcaaaaatgaagaaaaagaagaggatgacgcagagaattacagactctagctgctgcctgggggcgagcagcctaccagatgtcgatttgcctacgtggagcttcttatataggtactcttttgtctttacagaatgacgatacaaatgccaatgaccagatgtgacttattttccttttactatacagcttggcagagaaaataaatatcatcaaataagaaaaaa

SEQ ID NO: 7 - protein, 160 aa, Homo sapiens
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGG

SEQ ID NO: 8 - DNA, 480 bp, Homo sapiens
atgtggatcccagtggtcgggcttcctcggcggctgaggctctccgccttggcgggcgctggtcgcttttgcattttagggtctgaagcggcgacgcgaaagcatttgccggcgaggaaccactgtgggctctctgactcctctccgcagctgtggcccgaaccggatttcaggaatccgccaaggaaggcgtctaaggccagcttagactttaagcgttacgtaaccgatcggagattggctgagaccctggcgcaaatctatttgggaaaaccaagtagacctccacacctactgctggagtgcaatccaggtcctggaatcctgactcaggcattacttgaagctggtgccaaagtggttgcgctcgaaagtgacaaaacttttattccacatttggagtccttaggaaaaaatctggatggaaaactacgagtgattcactgtgacttctttaaactagatcctagaagtggtgga

SEQ ID NO: 9 - protein, 148 aa, Homo sapiens
MFRRPVLQVLRQFVRHESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 10 - DNA, 628 bp, Homo sapiens
cctgcgtggctgggctgctcgggttagatcgtcaggaaaagcctaaagattagactgtaagaaaagaaaatagaagccatgtttcgaagacctgtattacaggtacttcgtcagtttgtaagacatgagtccgaaacaactaccagtttggttcttgaaagatccctgaatcgtgtgcacttacttgggcgagtgggtcaggaccctgtcttgagacaggtggaaggaaaaaatccagtcacaatattttctctagcaactaatgagatgtggcgatcaggggatagtgaagtttaccaactgggtgatgtcagtcaaaagacaacatggcacagaatatcagtattccggccaggcctcagagacgtggcatatcaatatgtgaaaaaggggtctcgaatttatttggaagggaaaatagactatggtgaatacatggataaaaataatgtgaggcgacaagcaacaacaatcatagctgataatattatatttctgagtgaccagacgaaagagaaggagtagaaaggatgattcttctttggccatcatttggtacagtctcatttccaagtcatgtataatctttatggcttccaaggacaagaattaaaatactcttttacgt

SEQ ID NO: 11 - protein, 124 aa, Homo sapiens
MPAPAATYERVVYKNPSEYHYMKVCLEFQDCGVGLNAAQFKQLLISAVKDLFGEVDAALPLDILTYEEKTLSAILRICSSGLVKLWSSLTLLGSYKGKKCAFRVIQVSPFLLALSGNSRELVLD

SEQ ID NO: 12 - DNA, 1567 bp, Homo sapiens
gaattcggcacgagggagaagccaaacgtaaagacaccaggagtttctcgggcccagctgtggctgctgccggggagccccaagccttggcggtccttgctgcgaataggagtctggtcaggcgtcaggctagtccgacgaagagtgggtgtgatcagcactggaaaagatgcctgcccctgctgccacatatgaaagagtagtttacaaaaacccttccgagtaccactacatgaaagtctgcctagaatttcaagattgtggagttggactgaatgctgcacagttcaaacagctgcttatttcggctgtgaaggacctgtttggggaggttgatgccgccttacctttggacatcctaacctatgaagagaagaccttgtcagccatcttgagaatatgtagcagtggtcttgtcaaattgtggagctctttgaccctgttaggatcctataaaggcaaaaaatgtgctttccgggtgattcaggtttctccatttcttcttgcattatctggtaatagtagggaactagtattggattgaatgaatagtcttccattttggaaacgttcatccactctcatatttattttttcggtgcctgcatgtttgaagactgaagcaggctaaaagctcttgatgaaatttgagggtgctgaagatgttcccactaatttccagccatcacctttggtggggtgggcttcggaggacagtctgtctgaacctgccagtgctgaccctgcagcactttcagcatatgcacatcaaagttggagaccgcgctgaacttaggagggccttcacacagactgatgtggctaccttctcagaattaacaggggatgttaatcctttgcatttgaatgaagactttgcaaaacacaccaagtttggaaatacaattgtacatggagttttgatcaacggacttatctcagctctcctaggaactaaaatgccagggccaggctgtgtatttctttcccaggaaattagctttccagcccctttatatattggagaagttgttttagcttctgcagaagtgaaaaagctgaagcggttcattgctattattgcagtgtcatgttctgtaatagaaagtaaaaagactgttatggaaggctgggttaaagttatggttccagaagcttccaaatcctgaaatagatgttttaaagatgcaacctcaaacaccaatgctgttgttaaagagcctatggggaattgctgctctttaccaaagaatggttgataggcccagaagcccatgttagttaggggaagggagcaggaagagggttgttcaaatgcccactttccagtttggccttatgctttatgcagacttgagtgtatgcaggatttcattatctgcctgggtttttttgtttgttttttgttttttaattcaagaagtaggctgggcccggtggctcatgcctgtaatcctggcactttgggaggctgcggcaggcggatcacttgaggtcaggagtccaagaccagcctggccaacatggtgaaaccccatctctaccaaaaaaaaaaaaaaaaaaaaaa

SEQ ID NO: 13 - protein, 140 aa, Homo sapiens
MAENREPRGAVEAELDPVEYTLRKRLPSRLPRRPNDIYVNMKTDFKAQLARCQKLLDGGARGQNACSEIYIHGLGLAINHAINIALQLQAGSFGSLQVAANTSTVELVDELEPETDTREPLTRIRNNSAIHIRVFRVTPK

SEQ ID NO: 14 - DNA, 878 bp, Homo sapiens
gcgcgggcagggccgcacgagctggctggctgcttgcacccacatccttctttctctgggacctggggtcgcggttacttgggctggccggcgaacccttgagtggcctggcggggagcgggcctcgcgcgcctggagggccctgtggaacgaagagaggcacacagcatggcagaaaaccgagagccccgcggtgctgtggaggctgaactggatccagtggaatacacccttaggaaaaggcttcccagccgcttgccccggagacccaatgacatttatgtcaacatgaagacggactttaaggcccagctggcccgctgccagaagctgctggacggaggggcccggggtcagaacgcgtgctctgagatctacattcacggcttgggcctggccatcaaccacgccatcaacatcgcgctgcagctgcaggcgggcagcttcgggtccttgcaggtggctgccaatacctccaccgtggagcttgttgatgagctggagccagagaccgacacacgggagccactgactcggatccgcaacaactcagccatccacatccgagtcttcagggtcacacccaagtaattgaaaagacactcctccacttatcccctccgtgatatggctcttcgcatgctgagtactggacctcggaccagagccatgtaagaaaaggcctgttccctggaagcccaaaggactctgcattgagggtgggggtaattgtctcttggtggcccagttagtgggccttcctgagtgtgtgtatgcggtctgtaactattgccatataaataaaaaatcctgttgcactagtgtcctgccaaaaaaaaaaaaaaaaaaaaaaaaaaaaaaaaaaaaaaaaaaaaaaa

SEQ ID NO: 15 - protein, 220 aa, Homo sapiens [sequence omitted]

SEQ ID NO: 16 - DNA, 1130 bp, Homo sapiens
ctagagagcgccggaagcggtccgagaatgaagagtgtgatctaccatgcattgtctcagaaagaggcgaatgactccgatgtccagccttcaggagcacagcgggccgaggccttcgtgagggccttcctgaagcgcacgtcgccccgcatgagcccgcaggcccgcgaggaccagctgcagcgcaaggcggtggtcctggagtacttcacccgccacaagcgcaaggagaagaagaagaaagccaaaggcctctctgccaggcaaaggagggagctgcggctctttgacattaaaccagagcagcagagatacagccttttcctccctctccatgaactctggaaacagtacatcagggacctgtgcagtgggctcaagccagacacgcagccacagatgattcaggccaagctcttaaaggcagatcttcacggggctattatttcagtgacaaaatccaaatgcccctcttatgtgggtattacaggaatccttctacaggaaacaaagcacattttcaaaattatcaccaaagaagaccgcctgaaagttatccccaagctaaactgcgtgttcactgtggaaaccgatggctttatttcctacatttacgggagcaaattccagcttcggtcaagtgaacggtctgcgaagaagttcaaagcgaagggaacgattgacctgtgaattctttgccgtctaaggcagttgtttatgacagctgaaaactggacactccctaaatgtccacctttcagtgaagagatagttaagccaattccatttatagaccacctccagccagtgacgctccgagttgaggatgttgaacaacatgggaaggtcgcagcgtactaagtgaagaagtcagaggacagaggaatttctctttctaggagattttcattttgtgtgactcccatggggaggaacagactggcaggaagcacaccggggttaacactggttgacttgaataggattattcgatttttaaaaatacttttccatgttttctgagtgctctatgataaatcagttgcatctgtgataatacagtacatatgtggacataaacagggatcaaataaaggaggtattgctgcaaaaaaaaaaaaaaaaaaaaa

SEQ ID NO: 17 - protein, 268 aa, Homo sapiens
MAVFADLDLRAGSDLKALRGLVETAAHLGYSVVAINHIVDFKEKKQEIEKPVAVSELFTTLPIVQGKSRPIKILTRLTIIVSDPSHCNVLRATSSRARLYDVVAVFPKTEKLFHIACTHLDVDLVCITVTEKLPFYFKRPPINVAIDRGLAFELVYSPAIKDSTMRRYTISSALNLMQICKGKNVIISSAAERPLEIRGPYDVANLGLLFGLSESDAKAAVSTNCRAALLHGETRKTAFGIISTVKKPRPSEGDEDCLPASKKAKCEG

SEQ ID NO: 18 - DNA, 942 bp, Homo sapiens
gaattcggcacgaggtgggacttcagcatggcggtgtttgcagatttggacctgcgagcgggttctgacctgaaggctctgcgcggacttgtggagacagccgctcaccttggctattcagttgttgctatcaatcatatcgttgactttaaggaaaagaaacaggaaattgaaaaaccagtagctgtttctgaactcttcacaactttgccaattgtacagggaaaatcaagaccaattaaaattttaactagattaacaattattgtctcggatccatctcactgcaatgttttgagagcaacttcttcaagggcccggctctatgatgttgttgcagtttttccaaagacagaaaagctttttcatattgcttgcacacatttagatgtggatttagtctgcataactgtaacagagaaactaccattttacttcaaaagacctcctattaatgtggcgattgaccgaggcctggcttttgaacttgtctatagccctgctatcaaagactccacaatgagaaggtatacaatttccagtgccctcaatttgatgcaaatctgcaaaggaaagaatgtaattatatctagtgctgcagaaaggcctttagaaataagagggccatatgacgtggcaaatctaggcttgctgtttgggctctctgaaagtgacgccaaggctgcggtgtccaccaactgccgagcagcgcttctccatggagaaactagaaaaactgcttttggaattatctctacagtgaagaaacctcggccatcagaaggagatgaagattgtcttccagcttccaagaaagccaagtgtgagggctgaaaagaatgccccagtctctgtcagcactcccttcttcccttttatagttcatcagccacaacaaaaataaaacctttgtgtgaaaaaaaaaaaaaaaaaaaaaaaaaa

SEQ ID NO: 19 - protein, 283 aa, Homo sapiens
MAAAPQAPGRGSLRKTRPLVVKTSLNNPYIIRWSALESEDMHFILQTLEDRLKAIGLQKIEDKKKKNKTPFLKKESREKCSIAVDISDNLKEKKTDAKQQVSGWTPAHVRKQLVIGVNEVTRALERRELLLVLVCKSVKPAMITSHLIQLSLSRSVPACQVPRLSERIAPVIGLKCVLALAFKKNTTDFVDEVRAIIPRVPSLSVPWLQDRIEDSGENLETEPLESQDRELLDTSFEDLSKPKRKLADGRQASVTLQPLKIKKLIPNPNKIRKPPKSKKATPK

SEQ ID NO: 20 - DNA, 991 bp, Homo sapiens
aacggattcgccatcgtgagttccaggattttcaaaatggctgcagctcctcaagcaccggggcggggatctctccgtaagacgagacctctggttgtgaagacgtcgttgaacaacccatacatcatccgctggagcgctctggagagcgaggatatgcacttcatcctacagacgcttgaggacaggcttaaagctattggacttcagaagattgaagataagaagaaaaagaacaaaacaccttttctgaaaaaagaaagcagagagaaatgcagcattgctgttgatattagtgataatctgaaggagaagaaaacagatgctaagcagcaagtgtcagggtggacgcctgcacacgtcaggaagcagcttgtcattggcgttaacgaagttaccagagccctggaaaggagggaactgctgttagttctggtgtgtaaatcagtcaagcctgccatgatcacctcacacttgattcagttaagcctaagcagaagtgtccctgcctgtcaggtcccccggctcagtgagagaatcgcccccgtcattggcttaaaatgtgttctagccttggcgttcaaaaagaacaccactgactttgtggacgaagtaagagccatcatccccagagtccccagtttaagtgtaccatggcttcaagacagaattgaagattctggggaaaatttagagactgaacctctggaaagccaagacagagagcttttggacacttcatttgaagatctgtcaaaacctaagagaaagcttgctgacggtcggcaggcttctgtaacattacaaccccttaaaataaagaaactgattccaaaccctaataagataaggaaaccacccaaaagtaaaaaagctactccaaagtaatcttgcataaacttgtcatgtcatacagtttgtgaaaggacaccttgtaaagaagccttgaaactaataaaatgagttatacttacataaaaaaaaaaaaaaa

SEQ ID NO: 21 - protein, 302 aa, Homo sapiens
MNTGPYYFVKNLPLHELITPEFISTFIKKGSCYALTYNTHIDEDNTVALLPNGKLILSLDKDTYEETGLQGHPSQFSGRKIMKFIVSIDLMELSLNLDSKKYERISWSFKEKKPLKFDFLLAWHKTGSEESTMMSYFSKYQIQEHQPKVALSTLRDLQCPVLQSSELEGTPEVSCRALELFDWLGAVFSNVDLNEPNNFISTYCCPEPSTVVAKAYLCTITGFILPEKICLLLEHLCHYFDEPKLAPWVTLSVQGFADSPVSWEKNEHGFRKGGEHLYNFVIFNNQDYWLQMAVGANDHCPP

SEQ ID NO: 22 - DNA, 1194 bp, Homo sapiens
cggacggaggaaaagtggcgttgaaaaggcaagagccaggttcccagccactgggacagtgaatttacagcttcagacatccaccaaagagcggctgcgtgggctcctaagaagtctaagaactgctcagaaatccagctccgccatccccactccaccgccgccaggtcacagcagcgtgtttcatttctcattcctgaatgtgggatactatcggaagaactgaaaaacctggtcatgaacactggaccctattactttgtgaagaatttacctcttcatgaattaattacacctgaattcatcagtacctttataaagaaaggttcttgctatgcactaacatacaatacacatattgatgaagataatactgttgccctgctaccaaatgggaaattaattttgtcactggataaagacacttatgaagaaactggacttcagggtcatccatctcagttttctggcagaaaaattatgaaatttattgtttccattgatttgatggaattatccttaaacttggattctaagaagtatgaaagaatatcttggtctttcaaagaaaagaagccattgaaatttgattttcttttggcttggcataaaacaggttcagaagaatcgacaatgatgtcatatttttccaagtaccaaattcaggagcaccagccaaaagtagcactgagcacgttgagagatctccagtgcccagtgctgcagagcagcgagctggagggaacgccagaggtgtcctgccgggctctggagctcttcgactggctcggcgccgtcttcagtaatgtcgacctaaatgagcctaataatttcatatcaacctattgctgtcctgagccaagcacagtggtggcaaaagcttatttgtgtacaatcactggcttcatacttccagagaagatctgtctcctattggaacatctctgtcactactttgatgaaccgaagttagctccatgggttacactgtccgttcaaggctttgcagacagccctgtttcttgggaaaaaaatgaacatggttttcgaaaaggaggagaacatttatataactttgtgatttttaataatcaggactattggcttcagatggctgttggggcaaatgatcactgtccaccataaaaaaaaaaaaaaaaaaatcgtgtttacttacaaaaaaaaaaaaaaaaa

SEQ ID NO: 23 - protein, 903 aa, Homo sapiens
VTQKSSNSLVFQTLPRHMRRRAMSHNVKRLPRRLQEIAQKEAEKAVHQKKEHSKNKCHKARRCHMNRTLEFNRRQKKNIWLETHIWHAKRFHMVKKWGYCLGERPTVKSHRACYRAMTNRCLLQDLSYYCCLELKGKEEEILKALSGMCNIDTGLTFAAVHCLSGKRQGSLVLYRVNKYPREMLGPVTFIWKSQRTPGDPSESRQLWIWLHPTLKQDILEEIKAACQCVEPIKSAVCIADPLPTPSQEKSQTELPDEKIGKKRKRKDDGENAKPIKKIIGDGTRDPCLPYSWISPTTGIIISDLTMEMNRFRLIGPLSHSILTEAIKAASVHTVGEDTEETPHRWWIETCKKPDSVSLHCRQEAIFELLGGITSPAEIPAGTILGLTVGDPRINLPQKKSKALPNPEKCQDNEKVRQLLLEGVPVECTHSFIWNQDICKSVTENKISDQDLNRMRSELLVPGSQLILGPHESKIPILLIQQPGKVTGEDRLGWGSGWDVLLPKGWGMAFWIPFIYRGVRVGGLKESAVHSQYKRSPNVPGDFPDCPAGMLFAEEQAKNLLEKYKRRPPAKRPNYVKLGTLAPFCCPWEQLTQDWESRVQAYEEPSVASSPNGKESDLRRSEVPCAPMPKKTHQPSDEVGTSIEHPREAEEVMDAGCQESAGPERITDQEASENHVAATGSHLCVLRSRKLLKQLSAWCGPSSEDSRGGRRAPGRGQQGLTREACLSILGHFPRALVWVSLSLLSKGSPEPHTMICVPAKEDFLQLHEDWHYCGPQESKHSDPFRSKILKQKEKKKREKRQKPGRASSDGPAGEEPVAGQEALTLGLWSGPLPRVTLHCSRTLLGFVTQGDFSMAVGCGEALGFVSLTGLLDMLSSQPAAQRGLVLLRPPASLQYRFARIAIEV

SEQ ID NO: 24 - DNA, 4276 bp, Homo sapiens
ctgtgacccagaagtcttcgaattcactggtttttcagactctgccacggcacatgcgacgaagagccatgagccacaacgtcaaacgccttcccagacggttacaggagattgcccagaaagaggcggagaaagccgtacatcagaaaaaagaacattcaaaaaataaatgccataaagctcgaagatgtcacatgaaccggacgctagaatttaaccgtagacaaaagaagaacatttggttagaaactcacatctggcacgccaagcggtttcatatggtcaagaagtggggctactgccttggggagaggccaacagtcaagagccacagagcctgctatcgagccatgacgaaccggtgcctcctgcaggatttatcctattactgttgtttggagttgaaaggcaaagaggaagaaatactaaaggcgctttctggaatgtgtaacatagacacagggctgacgtttgcagcagttcactgcttgtctggaaagcgccaagggagccttgtgctttatcgggtgaataaatatcccagagaaatgcttgggcctgttacgtttatctggaagtcccagaggaccccgggtgacccttctgagagcaggcagctgtggatctggctgcatccaacccttaaacaggatatcttagaggaaataaaagcagcgtgccagtgtgtggaacccatcaaatcagctgtctgcatcgctgacccacttccaacaccatcccaagaaaaaagccaaactgaattgcctgacgagaaaattggcaagaaaagaaaaaggaaagatgatggagaaaatgctaaaccaattaaaaaaattatcggtgatggaactagagatccatgtctaccatactcttggatctctccaaccacaggcattataatcagcgatttgacgatggagatgaacagattccggctgattgggccactttcccactccatcctaactgaagcaataaaagctgcttctgtccacactgtgggagaggacacagaggagacacctcaccgctggtggatagaaacctgtaagaaacctgacagcgtttcccttcattgcagacaagaagccattttcgagttgttgggaggaataacatcaccagcagaaattccggcaggtactattctgggactgacagttggggatcctcgaataaatttgccccaaaagaagtccaaagctttgcccaatccagaaaaatgccaagataatgagaaagttagacagctgcttctggagggtgtgcctgtggaatgtacgcatagctttatctggaaccaagatatctgtaagagtgtcacagagaataaaatctcggatcaggatttaaaccggatgaggagtgaattgctggtgcctgggtcacagcttattttaggtccccatgaatccaagatacctatacttttgattcagcagccaggaaaagtgactggtgaagatcgactaggctggggaagtggctgggatgtcctactcccaaagggctggggcatggctttctggattccatttatttatcgaggtgtgagagtcggagggttgaaagagtctgcagtgcattctcagtataagaggtcgcctaatgtcccaggcgattttccagactgccctgccgggatgctgtttgcggaagagcaagctaagaatcttcttgaaaagtacaaaagacgccctcctgcaaaacggcccaactacgttaagcttggcactctggcacctttctgctgtccctgggagcagttaactcaagactgggagtcaagagtccaggcttacgaagaaccttctgtagcttcatctccaaatggtaaggagagtgacctaagaagatctgaggtgccttgtgctcccatgcctaaaaaaactcatcagccatctgatgaagtgggcacatccatagagcaccccagggaggcagaggaggtaatggatgcagggtgtcaagaatcggcagggcctgagaggatcacagaccaggaggccagtgaaaaccatgttgctgccacagggagtcacctctgcgttctcaggagtagaaaattactgaagcaactgtcagcctggtgtgggcccagttctgaggatagtcggggaggccggcgagctcccggcagaggccagcaaggattgaccagagaggcttgcctgtccatcttgggccacttccccagggccctggtttgggtcagcctgtccctgctcagcaagggcagccccgagcctcacaccatgatctgtgtcccagccaaggaggacttcctccagctccatgaggactggcattactgtgggccccaggaatccaaacacagtgacccattcaggagcaagatcctgaaacagaaagagaagaagaaaagggagaagaggcagaagccaggacgtgcctcttctgatggcccggcgggggaagagcccgtggctgggcaggaagctctgactctagggctgtggtcaggccctctgccgcgtgtgacgttgcactgctccagaactctcctaggctttgtgactcagggagatttttccatggctgttggctgtggagaagccctggggtttgttagcttgacaggcttgctggatatgctgtccagccagcctgcagcgcagaggggcttagtgctactgaggcctcccgcctctctgcagtatcgatttgcgaggattgctattgaggtgtgaatgcgtgcttgtatcccagcagggcatagataatacgttattattgtctgccaagttctacatgtggagaatctgcttctgctttaaaatatcatgtgaaactccctggaaacaagaataaaaaattatgtattatgcagatgatgaaatgtttacatcattccagtaatgtcattgattttcatctttccctgtccttgctgtaatacttttaaattatttggccaaaagctttgtattatgatctcttggtctgtgtagttgtggctgaaaataatgagaagctctacgagttatcatcccctttttttgttagaaacaaagggcttgtcaggtctatttgaaaaacctcatagtcatgtgataagcaacaatagatgtttaatgatttcactgttatagcagaagacaagagaagacgcttggcctctgtacatgaaatatgggctcctgatggacctcattcaattctgtactgtgatttccatgccgaacaactcaagccttaaagagagaaatcatggacaactgatttctgcctgttttcaggcaggcacagtttatggcgtcagtgctaggctggaattagaaagtgggggtctatgacgtggacttcctgactctttgatctctttgttgttgaccaacacttgatcctactagttacttaatttttttaagtaaaaaattattattattttgtttctgcaaagattttctcaaagccatagaggagcatttctcagaatatgttctatgatatgtgtcacctaaaaaagtaagagattccaaggtcaggttgatatggaaactctaggttaaataaagttaagcatttctttatgaaagaacttctggaaacttccatgtgataatgtgcattgcggatctctaggaaggaaatgatagtgtatagtattttctaaatacttgtgattcctaaagttctcttacaaggagccctttgtaggaccagtgttcttagtagcgcgctttgggcagtgtggctgtgtagtgcatagctacctctgcaaggtgataactaagccggcaagctgcctttcaacactcatgcagtcacgttgtccacctgagattctcaacagggtataaaaggaaggtctcatcttgcctcacaggaagagtgggctcagtgtggcttttttccaactatggagaaactcagtgctcatctactttaagtttccacatatggcttgctcatagccttggtccttacctttcctgccataactttctagaagagcttaatgggatttttttctaaaaaatgtaaatatgcagttaggcattattttatgtaaatgcattgggtttttactgtagcatttggcactaaatggctttgggggtgatgaggtggggaaggatacagcaggtggtacagtagtcaggaagtacctgccaccaatgagatgtctgatgctttgcctcttaccatgcctctgaatgtctttggatccaacccagatgagactgaaaaaaaaaaaacagtgtaactaagtggcatctgtaaacagaataaatgaaaatgtcacctg

SEQ ID NO: 25 - protein, 1239 aa, Homo sapiens
MSRLLWRKVAGATVGPGPVPAPGRWVSSSVPASDPSDGQRRRQQQQQQQQQQQQQPQQPQVLSSEGGQLRHNPLDIQMLSRGLHEQIFGQGGEMPGEAAVRRSVEHLQKHGLWGQPAVPLPDVELRLPPLYGDNLDQHFRLLAQKQSLPYLEAANLLLQAQLPPKPPAWAWAEGWTRYGPEGEAVPVAIPEERALVFDVEVCLAEGTCPTLAVAISPSAWYSWCSQRLVEERYSWTSQLSPADLIPLEVPTGASSPTQRDWQEQLVVGHNVSFDRAHIREQYLIQGSRMRFLDTMSMHMAISGLSSFQRSLWIAAKQGKHKVQPPTKQGQKSQRKARRGPAISSWDWLDISSVNSLAEVHRLYVGGPPLEKEPRELFVKGTMKDIRENFQDLMQYCAQDVWATHEVFQQQLPLFLERCPHPVTLAGMLEMGVSYLPVNQNWERYLAEAQGTYEELQREMKKSLMDLANDACQLLSGERYKEDPWLWDLEWDLQEFKQKKAKKVKKEPATASKLPIEGAGAPGDPMDQEDLGPCSEEEEFQQDVMARACLQKLKGTTELLPKRPQHLPGHPGWYRKLCPRLDDPAWTPGPSLLSLQMRVTPKLMALTWDGFPLHYSERHGWGYLVPGRRDNLAKLPTGTTLESAGVVCPYRAIESLYRKHCLEQGKQQLMPQEAGLAEEFLLTDNSAIWQTVEELDYLEVEAEAKMENLRAAVPGQPLALTARGGPKDTQPSYHHGNGPYNDVDIPGCWFFKLPHKDGNSCNVGSPFAKDFLPKMEDGTLQAGPGGASGPRALEINKMISFWRNAHKRISSQMVVWLPRSALPRAVIRHPDYDEEGLYGAILPQVVTAGTITRRAVEPTWLTASNARPDRVGSELKAMVQAPPGYTLVGADVDSQELWIAAVLGDAHFAGMHGCTAFGWMTLQGRKSRGTDLHSKTATTVGISREHAKIFNYGRIYGAGQPFAERLLMQFNHRLTQQEAAEKAQQMYAATKGLRWYRLSDEGEWLVRELNLPVDRTEGGWISLQDLRKVQRETARKSQWKKWEVVAERAWKGGTESEMFNKLESIATSDIPRTPVLGCCISRALEPSAVQEEFMTSRVNWVVQSSAVDYLHLMLVAMKWLFEEFAIDGRFCISIHDEVRYLVREEDRYRAALALQITNLLTRCMFAYKLGLNDLPQSVAFFSAVDIDRCLRKEVTMDCKTPSNPTGMERRYGIPQGEALDIYQIIELTKGSLEKRSQPGP

SEQ ID NO: 26 - DNA, 4465 bp, Homo sapiens
gcggaccggccgggtggaggccacacgctaccccgaggctgcgtaggccgcgcgaagggggacgccgtgccgtgggcctggggtcgggggagcagcagaccgggaagcaccgtgaggaccgaggatttggggtggaaggcaggcatggtcaaacccatttcactgacaggagagcagagacaggacgtgtctctctccacgtcttccagccagtaaaagaagccaagctggagcccaaagccaggtgttctgactcccagcgtgggggtccctgcaccaaccatgagccgcctgctctggaggaaggtggccggcgccaccgtcgggccagggccggttccagctccggggcgctgggtctccagctccgtccccgcgtccgaccccagcgacgggcagcggcggcggcagcagcagcagcagcagcagcagcagcagcaacagcagcctcagcagccgcaagtgctatcctcggagggcgggcagctgcggcacaacccattggacatccagatgctctcgagagggctgcacgagcaaatcttcgggcaaggaggggagatgcctggcgaggccgcggtgcgccgcagcgtcgagcacctgcagaagcacgggctctgggggcagccagccgtgcccttgcccgacgtggagctgcgcctgccgcccctctacggggacaacctggaccagcacttccgcctcctggcccagaagcagagcctgccctacctggaggcggccaacttgctgttgcaggcccagctgcccccgaagcccccggcttgggcctgggcggagggctggacccggtacggccccgagggggaggccgtacccgtggccatccccgaggagcgggccctggtgttcgacgtggaggtctgcttggcagagggaacttgccccacattggcggtggccatatccccctcggcctggtattcctggtgcagccagcggctggtggaagagcgttactcttggaccagccagctgtcgccggctgacctcatccccctggaggtccctactggtgccagcagccccacccagagagactggcaggagcagttagtggtggggcacaatgtttcctttgaccgagctcatatcagggagcagtacctgatccagggttcccgcatgcgtttcctggacaccatgagcatgcacatggccatctcagggctaagcagcttccagcgcagtctgtggatagcagccaagcagggcaaacacaaggtccagccccccacaaagcaaggccagaagtcccagaggaaagccagaagaggcccagcgatctcatcctgggactggctggacatcagcagtgtcaacagtctggcagaggtgcacagactttatgtaggggggcctcccttagagaaggagcctcgagaactgtttgtgaagggcaccatgaaggacattcgtgagaacttccaggacctgatgcagtactgtgcccaggacgtgtgggccacccatgaggttttccagcagcagctaccgctcttcttggagaggtgtccccacccagtgactctggccggcatgctggagatgggtgtctcctacctgcctgtcaaccagaactgggagcgttacctggcagaggcacagggcacttatgaggagctccagcgggagatgaagaagtcgttgatggatctggccaatgatgcctgccagctgctctcaggagagaggtacaaagaagacccctggctctgggacctggagtgggacctgcaagaatttaagcagaagaaagctaagaaggtgaagaaggaaccagccacagccagcaagttgcccatcgagggggctggggcccctggtgatcccatggatcaggaagacctcggcccctgcagtgaggaggaggagtttcaacaagatgtcatggcccgcgcctgcttgcagaagctgaaggggaccacagagctcctgcccaagcggccccagcaccttcctggacaccctggatggtaccggaagctctgcccccggctagacgaccctgcatggaccccgggccccagcctcctcagcctgcagatgcgggtcacacctaaactcatggcacttacctgggatggcttccctctgcactactcagagcgtcatggctggggctacttggtgcctgggcggcgggacaacctggccaagctgccgacaggtaccaccctggagtcagctggggtggtctgcccctacagagccatcgagtccctgtacaggaagcactgtctcgaacaggggaagcagcagctgatgccccaggaggccggcctggcggaggagttcctgctcactgacaatagtgccatatggcaaacggtagaagaactggattacttagaagtggaggctgaggccaagatggagaacttgcgagctgcagtgccaggtcaacccctagctctgactgcccgtggtggccccaaggacacccagcccagctatcaccatggcaatggaccttacaacgacgtggacatccctggctgctggtttttcaagctgcctcacaaggatggtaatagctgtaatgtgggaagcccctttgccaaggacttcctgcccaagatggaggatggcaccctgcaggctggcccaggaggtgccagtgggccccgtgctctggaaatcaacaaaatgatttctttctggaggaacgcccataaacgtatcagctcccagatggtggtgtggctgcccaggtcagctctgccccgtgctgtgatcaggcaccccgactatgatgaggaaggcctctatggggccatcctgccccaagtggtgactgccggcaccatcactcgccgggctgtggagcccacatggctcaccgccagcaatgcccggcctgaccgagtaggcagtgagttgaaagccatggtgcaggccccacctggctacacccttgtgggtgctgatgtggactcccaagagctgtggattgcagctgtgcttggagacgcccactttgccggcatgcatggctgcacagcctttgggtggatgacactgcagggcaggaagagcaggggcactgatctacacagtaagacagccactactgtgggcatcagccgtgagcatgccaaaatcttcaactacggccgcatctatggtgctgggcagccctttgctgagcgcttactaatgcagtttaaccaccggctcacacagcaggaggcagctgagaaggcccagcagatgtacgctgccaccaagggcctccgctggtatcggctgtcggatgagggcgagtggctggtgagggagttgaacctcccagtggacaggactgagggtggctggatttccctgcaggatctgcgcaaggtccagagagaaactgcaaggaagtcacagtggaagaagtgggaggtggttgctgaacgggcatggaaggggggcacagagtcagaaatgttcaataagcttgagagcattgctacgtctgacataccacgtaccccggtgctgggctgctgcatcagccgagccctggagccctcggctgtccaggaagagtttatgaccagccgtgtgaattgggtggtacagagctctgctgttgactacttacacctcatgcttgtggccatgaagtggctgtttgaagagtttgccatagatgggcgcttctgcatcagcatccatgacgaggttcgctacctggtgcgggaggaggaccgctaccgcgctgccctggccttgcagatcaccaacctcttgaccaggtgcatgtttgcctacaagctgggtctgaatgacttgccccagtcagtcgcctttttcagtgcagtcgatattgaccggtgcctcaggaaggaagtgaccatggattgtaaaaccccttccaacccaactgggatggaaaggagatacgggattccccagggtgaagcgctggatatttaccagataattgaactcaccaaaggctccttggaaaaacgaagccagcctggaccatagcactgcctggaggctctgtatttgctcccgtggagcttcatcggggtggtgcaggctcccaaactcaggctttcagctgtgctttttgcaaaagggcttgcctaaggccagccatttttcagtagcaggacctgccaagaagattccttctaactgaaggtgcagttgaattcagtgggttcagaaccaagatgccaacatcggtgtggactacaggacaaggggcattgttgcttgttgggtaaaaatgaagcagaagccccaaagttcacattaactcaggcatttcatttattttttccttttcttcttggctggttctttgttctgtcccccatgctctgatgcagtgccctagaaggggaaagaattaatgctctaacgtgataaacctgctccaaggcagtggaaataaaaagaaggaaaaaaaagaaaaaaaaaaaaaaaaaaaaaaaaaaaaaat

SEQ ID NO: 27 - protein, 372 aa, Homo sapiens
MVDLGGGVHGAVFPVDALHHKPSPLLPGDSAFRLVSAETLREILQDKELSKEQLVAFLENVLKTSGKLRENLLHGALEHYVNCLDLVNKRLPYGLAQIGVCFHPVFDTKQIRNGVKSIGEKTEASLVWFTPPRTSNQWLDFWLRHRLQWWRKFAMSPSNFSSSDCQDEEGRKGTNFTTIFPWGKELIETLWNLGDHELLHMYPGNVSKLHGRDGRKNVVPCVLSVNGDLDRGMLAYLYDSFQLTENSFTRKKNLHRKVLKLHPCLAPIKVALDVGRGPTLELRQVCQGLFNELLENGISVWPGYLETMQSSLEQLYSKYDEMSILFTVLVTETTLENGLIHLRSRDTTMKEMMHISKLKDFLIKYISSAKNV

SEQ ID NO: 28 - DNA, 1594 bp, Homo sapiens
gccaagcttggcacgaggtggcacgaggggcttgttgggatccgttgagtgatgggagagtgtgctctttaacttcggagagagatgcgctctcgtgtagccgtcagggcctgccataaggtctgcaggtgcctgttgtctgggtttgggggtcgagtagatgcggggcagccggagctgttgacggaaaggagtagccccaaaggagggcatgtgaagtcgcacgcggactcgaggggaacggcgagcacccagaagcccccgggtctggagagggaagcgaggcgctgttagagatctgtcagagaaggcatttcctaagtggaagcaagcagcagcttagccgggattctcttctgagtgggtgccatcccggcttcggacccttgggcgtagagttgcggaagaacctggccgcagaatggtggacctcggtggtggtgttcacggagcggtattcccggtggacgccctccaccacaaaccaagccctttgctacccggggacagtgccttcaggttagtttctgcagaaactctacgcgaaatcttgcaagacaaagagctgagtaaggaacagctagtagcatttcttgagaacgtattaaaaacttctgggaaactacgggagaaccttcttcacggtgccttggaacactatgttaattgcctggatctggtaaacaagaggctaccttatggccttgctcagattggagtgtgttttcatcctgtttttgacactaagcagatacgaaatggtgttaaaagtattggtgagaagactgaagcttcgttagtatggtttactcctccgagaacttcaaaccagtggcttgatttctggttacgtcatcgactccagtggtggagaaagtttgccatgagtccatctaacttcagcagcagtgactgtcaggatgaagaaggccggaaaggaacaaactttactacaattttcccctggggaaaggagttaatagaaaccctgtggaacttaggagatcacgaacttttacacatgtatcctggcaatgtgtctaaattacatggccgagatggacgaaaaaatgtggttccttgtgttctctctgtaaatggggacctagaccgaggcatgctggcctacctctatgattctttccagctgacagagaactcctttacaagaaagaaaaatcttcatagaaaggtacttaaacttcacccttgtttagcccctattaaggttgctttggatgtaggaagaggccccacattggaactaagacaggtttgtcaagggctatttaatgagttactagaaaatgggatttctgtgtggcctggttatttggaaactatgcagtcctcattggaacaactttattcgaagtatgatgaaatgagtattctcttcacagttttggttactgaaactactttggagaatggattaatacatctgagaagcagagacaccacaatgaaggaaatgatgcatatatccaaattaaaagactttttgattaagtatatatcatcagctaagaatgtatagatttttatatttgtataataaatattcttctctcctaaaaaaaaaaaaaaaaaa

SEQ ID NO: 29 - protein, 10 aa, Artificial Sequence
  Description of Artificial Sequence poly-His tag
HHHHHHHHHH

SEQ ID NO: 30 - protein, 6 aa, Artificial Sequence
  Description of Artificial Sequence poly-His tag
HHHHHH

SEQ ID NO: 31 - protein, 313 aa, Caenorhabditis elegans
MASASRLPPLPALRDFIHMYRLRAKKILSQNYLMDMNITRKIAKHAKVIEKDWVIEIGPGPGGITRAILEAGASRLDVVEIDNRFIPPLQHLAEAADSRMFIHHQDALRTEIGDIWKNETARPESVDWHDSNLPAMHVIGNLPFNIASPLIIKYLRDMSYRRGVWQYGRVPLTLTFQLEVAKRLCSPIACDTRSRISIMSQYVAEPKMVFQISGSCFVPRPQVDVGVVRFVPRKTPLVNTSFEVLEKVCRQVFHYRQKYVTKGLKTLYPEELEDELSDDLLKKCRIDPTTTSIRLGIEQFADLAEGYNEQCIR

SEQ ID NO: 32 - protein, 396 aa, Homo sapiens
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGGVIKPPAMSSRGLFKNLGIEAVPWTADIPLKVVGMFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 33 - protein, 366 aa, Schizosaccharomyces pombe
MKLPKILYDAAAFGGPRSTGFVKILNLNGRSSYKSSYLVNQNLMDEALVKSNLLKEYNSEKMTILEMAPGPGVTTTSLFNYFQPKSHVVLESREVFSKPLQKLCTLSDGRIKWVHQDGYYWQTYEDVYVSKVLDPRIQTEEEQKLSPHRELLFFAHLPHGYAGLLFVSQILDFLSARDWLGIFGRVRVLLWLPCSPTVTLLGSRGFSKRSKTSVFREAFTDSRVLAASESTLQKLCMGYSKEAKENYQISPNPLLVSPTPITSEPHKEDLTLVEMCSKPQDKQLSIPVFESIVRILLTCKATSLSKSIYYLGPGAETLLPSFTQCGINIDMPVGLLSAADFLTISKIIQKYPFKHHLHLGTIIEDS

SEQ ID NO: 34 - protein, 341 aa, Saccharomyces cerevisiae
MSVPIPGIKDISKLKFFYGFKYLWNPTVYNKIFDKLDLTKTYKHPEELKVLDLYPGVGIQSAIFYNKYCPRQYSLLEKRSSLYKFLNAKFEGSPLQILKRDPYDWSTYSNLIDEERIFVPEVQSSDHINDKFLTVANVTGEGSEGLIMQWLSCIGNKNWLYRFGKVKMLLWMPSTTARKLLARPGMHSRSKCSVVREAFTDTKLIAISDANELKGFDSQCIEEWDPILFSAAEIWPTKGKPIALVEMDPIDFDFDVDNWDYVTRHLMILKRTPLNTVMDSLGHGGQQYFNSRITDKDLLKKCPIDLTNDEFIYLTKLFMEWPFKPDILMDFVDMYQTEHSG

SEQ ID NO: 35 - protein, 268 aa, Pseudomonas aeruginosa
MSELYQHRARKRFGQNFLHDAGVIHRILRAIHAREGQRLLEIGPGQGALTEGLLGSGARLDVIELDQDLIPLLKLKFGLESRFSLHQGDALKFDFASLVESGEKLRVVGNLPYNISTPLIFHLLEHAPVIEDMHFMLQKEVVERLAATPGGGDWGRLSIMVQYHCRVEHLFNVGPGAFNPPPKVDSAIVRLTPFAEPPHPARDPKLLERVVREAFNQRRKTLRNTLKPLLSVEDIEAAEVDPTLRPEQLDLAAFVRLANQLAELPGNR

SEQ ID NO: 36 - protein, 273 aa, Escherichia coli
MNNRVHQGHLARKRFGQNFLNDQFVIDSIVSAINPQKGQAMVEIGPGLAALTEPVGERLDQLTVIELDRDLAARLQTHPFLGPKLTIYQQDAMTFNFGELAEKMGQPLRVFGNLPYNISTPLMFHLFSYTDAIADMHFMLQKEVVNRLVAGPNSKAYGRLSVMAQYYCNVIPVLEVPPSAFTPPPKVDSAVVRLVPHATMPHPVKDVRVLSRITTEAFNQRRKTIRNSLGNLFSVEVLTGMGIDPAMRAENISVAQYCQMANYLAENAPLQES

SEQ ID NO: 37 - protein, 346 aa, Homo sapiens
MAASGKLSTCRLPPLPTIREIIKLLRLQAAKQLSQNFLLDLRLTDKIVRKAGNLTNAYVYEVGPGPGGITRSILNADVAELLVVEKDTRFIPGLQMLSDAAPGKLRIVHGDVLTFKVEKAFSESLKRPWEDDPPNVHIIGNLPFSVSTPLIIKWLENISCRDGPFVYGRTQMTLTFQKEVAERLAANTGSKQRSRLSVMAQYLCNVRHIFTIPGQAFVPKPEVDVGVVHFTPLIQPKIEQPFKLVEKVVQNVFQFRRKYCHRGLRMLFPEAQRLESTGRLLELADIDPTLRPRQLSISHFKSLCDVYRKMCDEDPQLFAYNFREELKRRKSKNEEKEEDDAENYRL

SEQ ID NO: 38 - protein, 345 aa, Mus musculus
MAASGKLGTFRLPPLPTIREIIKLFGLRAVKQLSQNFLLDLRLTDKIVRKAGSLADVYVYEVGPGPGGITRSILNANVAELLVVEKDTRFIPGLQMLSDAAPGKLRIVHGDVLTYKIEKAFPGNIRRQWEDDPPNVHIIGNLPFSVSTPLIIKWLENISLKDGPFVYGRTKMTLTFQKEVAERLVATTGSKQHSRLSIMAQYLCNVEHLFTIPGKAFVPKPKVDVGVVHLTPLIEPKIKQPFKLVEKVVQNAFQFRRKYCHRGLGMLFPEAQRLESTGRLLQLADIDPTLRPTHLSLMHFKSLCDVYRKMCDEDPQLFTYNFREELKQKKSKGQEKDGDPESCGF

SEQ ID NO: 39 - protein, 396 aa, Homo sapiens; identical to SEQ ID NO: 32

SEQ ID NO: 40 - protein, 396 aa, Mus musculus
MRGPAMRLPPRLALSALARGPSCILGSGAATRKDWQTRNGRGFSDFNIEPLPDSDLEESSPWTSRNRSEPTRHIACKKAARNLVRDLLEHQNPSRQIILECNPGPGILTGALLKAGARVVAFESEKTFIPHLEPLQRNMDGELQVVHCDFFKMDPRYQEVVRPDVSSQAIFQNLGIKAVPWSAGVPIKVFGILPYKHERRILWKILFDLYSCESIYRYGRVELNMFVSEKEFRKLIATPKRPDLYQVMAVLWQVACDVKFLHMEPWSSFSVHMENGHLEKSKHGESVNLLKQNLYLVRMTPRRTLFTENLSPLNYDIFFHLVKHCFGKRNAPIIRHLRSLSTVDPINILRQIRKNPGDTAARMYPHDFKKLFETIEQSEDSVFKWIYDYCPEDMEF

(19) United States
(12) Patent Application Publication
(10) Pub. No.: US 2004/0253728 A1
(43) Pub. Date: Dec. 16, 2004

(54) New method

(76) Inventors: Claes Gustafsson, Tullinge (SE); Nils-Goran Larsson, Huddinge (SE)

Correspondence Address: YOUNG & THOMPSON, 745 SOUTH 23RD STREET, 2ND FLOOR, ARLINGTON, VA, 22202, US

(21) Appl. No.: 10/416,456

(22) PCT Filed: Nov. 12, 2001
(86) PCT No.: PCT/SE01/02501

Related U.S. Application Data
(60) Provisional application No. 60/248,567, filed on Nov. 16, 2000.

(30) Foreign Application Priority Data
Nov. 10, 2000 (SE) 0004127.7

(51) Int. Cl. C12N 15/85

(52) U.S. Cl. 435/455

(57) ABSTRACT

Apoptosis can be induced in a mammalian cell by administering a substance capable of impairing mammalian mitochondrial DNA gene expression to said cell in such an amount that apoptosis is induced. Certain antisense nucleic acid molecules specifically binding to nucleic acid molecules encoding proteins affecting mitochondrial gene expression are preferably used. The invention also provides novel such antisense nucleic acid molecules and pharmaceutical compositions containing the novel compounds. The invention also describes the use of an in vitro assay consisting of TFAM, TFB1M, TFB2M, mtRNAP and a mtDNA promoter fragment, to identify substances that inhibit or stimulate mtDNA transcription.

The present invention relates to a new method for inducing apoptosis of a living mammalian cell. According to the invention, substances impairing mammalian mitochondrial DNA gene expression are administered to such cells thereby inducing apoptosis. The invention also provides novel substances capable of impairing mammalian mitochondrial DNA gene expression and pharmaceutical compositions containing such substances. The invention also include the identification of two essential factors for mammalian mitochondrial DNA gene expression and the development of an in vitro assay for high-throughput identification of inhibitors and stimulators of mammalian mitochondrial gene expression.

### TECHNICAL BACKGROUND

The process of apoptosis—that is, the normal physiological process of programmed cell death, maintains tissue homeostasis. Changes to the apoptotic pathway that prevent or delay normal cell turnover can be just as important in the pathogenesis of diseases as are abnormalities in the regulation of the cell cycle. Like cell division, which is controlled through complex interactions between cell cycle regulatory proteins, apoptosis is similarly regulated under normal circumstances by the interaction of gene products that either prevent or induce cell death.

Since apoptosis functions in maintaining tissue homeostasis in a range of physiological processes such as embryonic development, immune cell regulation and normal cellular turnover, the dysfunction or loss of regulated apoptosis can lead to a variety of pathological disease states. Diseases and conditions in which apoptosis has been implicated fall into two categories, those in which there is:

increased cell survival (i.e., apoptosis is reduced)

increased cell death (i.e., apoptosis is increased).

Mitochondria are small (0.5-1 μm) organelles located in the cytoplasm of all eukaryotic cells. The organelle contains an inner and an outer membrane, which defines the matrix and the intermembrane space. The outer membrane is permeable to small molecules (up to 10 kD) whereas the inner membrane is freely permeable to oxygen and carbon dioxide. This relative impermeability of the inner membrane is essential for maintaining a proton gradient required for the synthesis of adenosine triphosphate (ATP). The inner membrane is folded into cristae, which increases the membrane surface available for assembly of the respiratory chain enzyme complexes. The mitochondrial network of a cell contains between 10³-10⁴ copies of a closed circular DNA genome (mtDNA) with a molecular size of 16,569 basepairs (Anderson S, et al. Nature 1981; 290: 457-65). The mtDNA contains only 37 genes, of which 24 encode RNAs necessary for protein synthesis (22 tRNAs and 2 rRNAs) (Anderson et al. Nature 1981; 290: 457-65; Bibb et al. Cell 1981; 26: 167-180). The remaining 13 genes encode proteins that are critical subunits of the respiratory chain and thus have a key role in regulating oxidative phosphorylation. One can therefore assume that the exact levels of mtDNA gene expression will directly influence the respiratory status of the eukaryotic cell. The mtDNA is replicated and transcribed within the mitochondrial matrix (Clayton D A. Annu Rev Cell Biol 1991; 7:453-78). Initiation of transcription occurs at several promoters of the large Saccharomyces cerevisiae mtDNA and requires only two proteins, yeast mitochondrial RNA polymerase (mtRNA pol), Rpo41 (Masters et al. Cell 1991; 51:89-99), and its specificity factor, Mtf1 (Schinkel et al. J Biol Chem 1987; 262:12785-91; Shadel and Clayton Mol Cell Biol 1995; 15:2101-08). In contrast, transcription of mammalian mtDNA is dependent on the high mobility group-box protein TFAM (previously mtTFA) (Fisher and Clayton Mol Cell Biol 1988; 8:3496-509; Parisi and Clayton Science 1991; 252:965-969; Shadel and Clayton Annu Rev Biochem 1997; 66:409-35). Surprisingly, the yeast TFAM homologue, Abf2, does not activate transcription but rather functions as a mtDNA stability factor (Dairaghi et al. Bba-Mol Basis Dis 1995; 1271: 127-134; Diffley and Stillman, Proc Natl Acad Sci USA 1991; 88:7864-7868; Parisi et al. Mol Cell Biol 1993; 13:1951-1961). The compact mammalian mtDNA contains only two promoters, the light and heavy strand promoters (LSP and HSP), which produce near genomic length transcripts that are processed to yield the individual mRNAs, tRNAs and rRNAs. Transcription from LSP is not only necessary for gene expression but also produces an RNA primer required for initiation of mtDNA replication (Shadel and Clayton Annu Rev Biochem 1997; 66:409-35). Germ line disruption of the mouse Tfam gene leads to loss of mtDNA, severe respiratory chain deficiency and embryonic lethality, which is likely a consequence of abolished transcription-dependent priming of mtDNA replication (Larsson. et al. Nature Genet 1998; 18:231-236). Recombinant TFAM protein and a partially purified human mtRNAP fraction are sufficient for activation of LSP and HSP transcription in vitro (Dairaghi et al. Bba-Mol Basis Dis 1995; 1271: 127-134; Dairaghi et al. J Mol Biol 1995; 249:11-28; Fisher and Clayton Mol Cell Biol 1988; 8:3496-509; Fisher et al. Genes Dev 1989; 3; 2202-2217). However, experiments aimed at in vitro reconstitution of human mtDNA transcription with recombinant mtRNAP and TFAM proteins have been unsuccessful and it has been speculated that additional factors are required (Prieto-Marin et al. FEBS lett 2001; 503; 51-55; Tiranti et al. Hum Mol Genet 1997; 6:615-25).

The mitochondrion, which was once thought simply to generate energy for a cell, is, in fact, a pivotal decision center controlling apoptosis by releasing death-promoting factors into the cytosol. Cytochrome c, a mitochondrial protein that normally shuttles electrons between protein complexes in the inner mitochondrial membrane, can induce apoptosis when released to the cytosol. In the presence of ATP, cytosolic cytochrome c interacts directly with the apoptotic protease activating factor-1 (Apaf-1) and procaspase 9 to form the apoptosome. The apoptosome is a macromolecular complex that cleaves procaspase 9 to active caspase 9 (Li et al. Cell 1997; 91:479-489). Subsequently, caspase 9 cleaves procaspase 3 to active caspase 3. The mitochondrial release of cytochrome c can be controlled by the Bcl-2 family proteins and other factors. The Bcl-2 family proteins can prevent cell death by inhibiting release of cytochrome c (Bcl-2 and Bcl-xL) or promote cell death by inducing cytochrome c release (Bax and Bak). Apoptosis can further be induced by activation of death receptors. Binding of extracellular ligands, such as Fas ligand or TNFα, to their respective receptors induces receptor trimerization, which, in turn, recruits adaptor molecules, e.g. FADD and TRADD, and procaspase 8. This signalling complex activates procaspase 8 and downstream events include activation of procaspase 3 and also cytochrome c release mediated by cleavage of Bid (Nagata Cell 1997; 88:355-365; Luo et al. Cell 1998; 94:481-490). Both the mitochondrial and the death receptor pathways thus converge on cleavage of procaspase 3 resulting in DNA fragmentation after activation of CAD or DFF (Sakahira et al. Nature 1998; 391:96-99; Enari et al. Nature 1998; 391:43-50; Liu et al. Cell 1997; 89:175-184).

In other respects, an inhibition of a component of the mitochondrial pathway, the NADH dehydrogenase subunit 4 (ND4), by specific inhibitors of the mitochondrial pathway, namely Rotenone, Oligomycine and Antimycin A, has been shown to increase cell death in the cell population and to induce differentiation in the surviving population (Mills et al., Biochemical and Biophysical Research Communication 1999; 263:294-300).

Inhibition of the activity of a component of the mitochondrial pathway derived from a mitochondrial gene, namely cytochrome c oxidase/serine tRNA, by the use of an antisense RNA comprising both a sense serine tRNA portion and an antisense cytochrome c oxidase portion, and named MARCO, has also been shown to induce cell death (Shirafuji et al., Blood 1997; 90:4567-4577).

Diseases in which there is an excessive accumulation of cells due to increased cell survival are exemplified by, but not limited to, neoplasia, hyperproliferative syndromes, autoimmune disorders and viral infections. Until recently, it was thought that cytotoxic drugs killed target cells directly by interfering with some life-maintaining functions. However, of late, it has been shown that exposure to several cytotoxic drugs with disparate mechanisms of action induces apoptosis in both malignant and normal cells. Apoptosis is also essential for the removal of potentially autoreactive lymphocytes during development and the removal of excess cells after the completion of an immune or inflammatory response. Recent work has clearly demonstrated that improper apoptosis may underlie the pathogenesis of autoimmune diseases by allowing abnormal autoreactive lymphocytes to survive. Apoptosis is also believed to be relevant for regulating angiogenesis. Increased angiogenesis is found in neoplasia, because tumor cells release angiogenic factors recruiting endothelial cells to the tumor site, and also in numerous other conditions, e.g. diabetic retinopathy and retinopathy of preterm babies. It would therefore be desirable to sensitize angiogenic endothelial cells to apoptotic stimuli (e.g. chemotherapeutic drugs, radiation, or endogenous TNFα) to block angiogenesis in these conditions. Promotion of or sensitization to apoptosis is believed to have clinical relevance in, for example, sensitizing cancer cells to chemotherapeutic drugs or radiation.

The second category, i.e. excessive cell death, is exemplified by, but not limited by, the conditions described below. Increased apoptosis has been documented in AIDS, neurodegenerative disorders (e.g. Alzheimer's disease, Parkinson's disease, amyotrophic lateral sclerosis), heart failure and different types of diabetes mellitus. Apoptosis occurs in conditions characterized by ischemia, e.g. myocardial infarction and cerebral stroke. Apoptosis has also been implicated in a number of liver disorders, including obstructive jaundice and hepatic damage due to toxins and drugs, kidney disorders, e.g. polycystic kidney disease, and different disorders of the pancreas including diabetes. For these and other diseases and conditions in which unwanted apoptosis is believed to be involved, novel ways of inhibiting apoptosis are desired.

Clearly there is a need for compounds and methods, which are specifically designed to modulate apoptosis in order to treat a wide variety of human diseases. The present invention provides a novel method of regulating apoptosis by regulating mitochondrial gene expression. The unexpected findings that decreased mtDNA gene expression promotes apoptosis and that increased mtDNA gene expression inhibits apoptosis provide two novel avenues for modifying apoptosis in human disease.

There is also a need for substances which may stimulate mtDNA gene expression. Such substances could stimulate synthesis of the mitochondrially encoded components of the electron transport chain, thereby stimulating the respiratory status of the cell. Such stimulatory substances could be used for the treatment of a number of different human disorders, including obesitas.

This invention also describe the identification of new mitochondrial transcription factors and their use in an in vitro assay, developed to allow the identification of substances, which can inhibit or stimulate mtDNA gene expression.

### SUMMARY OF THE INVENTION

It has now turned out that apoptosis can be induced in a mammalian cell by administering a substance capable of impairing mammalian mitochondrial DNA gene expression to said cell in such an amount that apoptosis is induced. Certain antisense nucleic acid molecules specifically binding to nucleic acid molecules encoding proteins affecting mitochondrial gene expression are preferably used. The invention also provides novel such antisense nucleic acid molecules and pharmaceutical compositions containing the novel compounds. The invention also provides the identification of novel factors needed for mitochondrial transcription and a method in which these factors are used to identify substances with an inhibitory or stimulatory effect on mtDNA gene expression.

### DETAILED DESCRIPTION OF THE INVENTION

As already mentioned, the present invention relates to a method for inducing apoptosis of a living mammalian cell, comprising the steps of:

a) providing a substance capable of impairing mammalian mitochondrial DNA gene expression; and

b) administering said substance to said living mammalian cell in such an amount that apoptosis is induced.

Substances capable of impairing mammalian mitochondrial DNA gene expression are, among all, substances affecting the expression of nuclear genes regulating:

a) mitochondrial DNA replication;

b) mitochondrial DNA maintenance and stability;

c) mitochondrial DNA transcription;

d) processing and stability of mitochondrial transcripts;

e) mitochondrial protein translation; and/or

f) stability of mitochondrially encoded proteins.

Examples of such nuclear genes are genes encoding mitochondrial RNA polymerase (SEQ. ID. NO. 2), mitochondrial transcription factor A (mtTFA or TFAM) (SEQ. ID. NO. 4), mitochondrial single strand binding protein (mtSSB) (SEQ. ID. NO. 10), ribonucleotidase mitochondrial RNA processing (RNAse MRP) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), ribonucleotidase P (RNAse P) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), the catalytic or accessory subunit of mitochondrial DNA polymerase (SEQ. ID. NO. 26, SEQ. ID. NO. 28), and mammalian homologues of yeast Mtf1, herein referred to as TFB1M (SEQ. ID. NO. 6) and TFB2M (SEQ. ID. NO. 8).

Preferably, the induction of apoptosis is accomplished by antisense nucleic acid molecules.

In a preferred embodiment, the present invention employs oligomeric antisense compounds, particularly oligonucleotides, for use in modulating the function of nucleic acid molecules encoding factors affecting mitochondrial DNA gene expression, ultimately modulating the amount of such produced. The modulation of the function of selected nucleic acid molecules encoding these factors provides a flexible regulation of mitochondrial DNA gene expression, which permits the development of novel treatments of common human diseases associated with mitochondrial dysfunction, This is accomplished by providing antisense compounds which specifically hybridize with one or more nucleic acids encoding factors affecting mitochondrial DNA gene expression.

Among the factors affecting mitochondrial DNA gene expression, specific factors such as the transcription factors regulating mitochondrial DNA gene expression are of special interest. Some of these transcription factors have been identified and characterised, such as mitochondrial transcription factors B1, (TFB1M), B2 (TFB2M) and A (TFAM). These transcription factors have been shown to interact together and also with mitochondrial RNA processing ribonuclease (Rnase MRP) to activate mtDNA transcription (Falkenberg et al., unpublished results). Thus, the understanding of the interaction mechanism between these transcription factors and further proteins necessary for basal transcription of mammalian mitochondrial DNA provides novel pathways for therapeutic intervention in the large group of disorders associated with mitochondrial dysfunction and disclosed, for example, by D. C. Wallace (Science, 1999, 283:1482-1488) or by N. G. Larsson et al. (FEBS Letters, 1999, 455:199-202).

In the context of the present invention, the nucleic acid molecules encoding the above-mentioned transcription factors are only examples of suitable target molecules, and shall thus not be considered as a limitation of the scope of the invention to theses specific molecules.

As used herein, the term “target nucleic acid” encompass DNA encoding factors affecting mitochondrial DNA gene expression, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of factors affecting mitochondrial DNA gene expression. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding factors affecting mitochondrial DNA gene expression. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding factors affecting mitochondrial DNA gene expression, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′-untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

For example Lee et al. (PNAS, 1996, 93:11471-11476) have used antisense RNAs to identify the in situ association in a macromolecular complex, possibly 60-80S preribosomes, of two ribonucleoproteins, namely RNase mitochondrial RNA processing enzyme (MRP) and RNase P.

In other respects, Inagaki et al. (Biochemistry and Molecular Biology International, 1998, 45:567-573) have used antisense RNAs of the gene encoding mitochondrial transcription factor A to provide evidence of a control of mitochondrial gene expression by this transcription factor.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e. from about 8 to about 30 linked nucleosides). Particularly preferred antisense compounds are antisense oligonucleotides, even more preferably those comprising from about 12 to about 25 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Another useful oligotide mimetic is LNA [Wahlestedt et al., Proc. Natl. Acad. Sci. USA 97:5633-5638 (2000)]

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘ CH₃, O(CH₂)ₙOCH₃, O(CH₂)ₙNH₂, O(CH₂)ₙCH₃, O(CH₂)ₙ ONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃NH₂. heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylamimoethoxyethoxy (also known in the art as 2′-O-dimethylaminoethoxyethyl or 2′-DMAEOE), i.e., 2′OCH₂OCH₂N(CH₂)₂, also described in examples hereinbelow.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for in increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, and each of which is herein incorporated by reference, and U.S. Pat. No. 5,750,692, and also herein incorporated by reference.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, and each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921,; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediame, N-methylglucamine, and procaine (see, for example, Berge et al., “Pharmaceutical Salts,” J. of Pharma Sci. 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of methionine aminopeptidase 2 is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding methionine aminopeptidase 2, enabling sandwich and other assays to easily be constructed to exploit this fact. Hybridization of the antisense oligonucleotides of the invention with a nucleic acid encoding methionine aminopeptidase 2 can be detected by means known in the art. Such means may include conjugation of an enzyme to the oligonucleotide, radiolabelling of the oligonucleotide or any other suitable detection means. Kits using such detection means for detecting the level of methionine aminopeptidase 2 in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

### Emulsions

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988,Marcel Dekker, Inc., New York, N.Y., volume 1, p. 199; Rosoff in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1998, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotopically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and deceased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome.™. I (glyceryl dilaurate/cholesterol/polyoxyethylene-10-stearyl ether) and Novasome.™. II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G.sub.M1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G.sub.M1, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G.sub.M1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C.sub.12 15G, that contains a PEG moiety. Ilium et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) a described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add the edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isothionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

### Penetration Enhancers

In one embodiment, the present invention employs various penetration enhancers to effect the efficient delivery of nucleic acids, particularly oligonucleotides, to skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C.sub.1-10 alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate); glycodeoxycholic acid (sodium glycodeoxycholate), taurocholic acid (sodium taurocholate), taurodeoxcholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al,, J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

### Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases.

The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

### Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, anti-pruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

Accordingly, the present invention provides antisense nucleic acid molecules that is complementary to and/or specifically binds to a nucleic acid molecule, such as a DNA or an RNA molecule, encoding mitochondrial RNA polymerase (SEQ. ID. NO. 2), mitochondrial transcription factor A (mtTFA or TFAM) (SEQ. ID. NO. 4), mitochondrial single strand binding protein (mtSSB) (SEQ. ID. NO. 10), ribonucleotidase mitochondrial RNA processing (RNAse MRP) (SEQ. ID. NO. 12 , SEQ. ID. NO. 14, SEQ. ID. NO. 16, SEQ. ID. NO. 18, SEQ. ID. NO. 20, SEQ. ID. NO. 22, SEQ. ID. NO. 24), ribonucleotidase P (RNAse P) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), the catalytic or accessory subunit of mitochondrial DNA polymerase (SEQ. ID. NO. 26, SEQ. ID. NO. 28), and mammalian homologues of yeast Mtf1, herein referred to as TFB1M (SEQ. ID. NO. 6) and TFB2M (SEQ. ID. NO. 8).

The present invention also provides pharmaceutical compositions containing antisense nucleic acid molecules that is complementary to and/or specifically binds to a nucleic acid molecule, such as a DNA or an RNA molecule, encoding mitochondrial RNA polymerase (SEQ. ID. NO. 2), mitochondrial transcription factor A (mtTFA or TFAM) (SEQ. ID. NO. 4), mitochondrial single strand binding protein (mtSSB) (SEQ. ID. NO. 10), ribonucleotidase mitochondrial RNA, processing (RNAse MRP) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), ribonucleotidase P (RNAse P) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), the catalytic or accessory subunit of mitochondrial DNA polymerase (SEQ. ID. NO. 26, SEQ. ID. NO. 28), and mammalian homologues of yeast Mtf1, herein referred to as TFB1M (SEQ. ID. NO. 6) and TFB2M (SEQ. ID. NO. 8), together with a pharmaceutically acceptable carrier, excipient or diluent.

It should also be possible to impair mitochondrial DNA gene expression by directly affecting the function or activity of nuclear gene products regulating:

a) mitochondrial DNA replication;

b) mitochondrial DNA maintenance and stability;

c) mitochondrial DNA transcription;

d) processing and stability of mitochondrial transcripts;

e) mitochondrial protein translation; and/or

f) stability of mitochondrially encoded proteins.

These nuclear gene products are exemplified by, but not limited to, mitochondrial RNA polymerase (SEQ. ID. NO. 2), mitochondrial transcription factor A (mtTFA or TFAM) (SEQ. ID. NO. 4), mitochondrial single strand binding protein (mtSSB) (SEQ. ID. NO. 10), ribonucleotidase mitochondrial RNA processing (RNAse MRP) (SEQ. ID. NO. 12, SEQ. ID. NO. 4, SEQ. ID. NO. 16, SEQ. ID. NO. 18, SEQ. ID. NO. 20, SEQ. ID. NO. 22, SEQ. ID. NO. 24), ribonucleotidase P (RNAse P) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), the catalytic or accessory subunit of mitochondrial DNA polymerase (SEQ. ID. NO. 26, SEQ. ID. NO. 28), and mammalian homologues of yeast Mtf1, herein referred to as TFB1M (SEQ. ID. NO. 6) and TFB2M (SEQ. ID. NO. 8). Suitable compounds capable of directly affecting the function or activity of the above nuclear gene products can by found by applying the method described in Example 6 below.

By administering substances capable of inducing apoptosis, and thereby inducing cell death, it should be possible to treat a human or an animal having a disease or a condition characterized by decreased cell death, exemplified by, but not limited to cancer, lymphoproliferative syndromes, autoimmune diseases, sarcomas, menigeomas, basal cell carcinomas, benign tumors, psoriasis, and prostatic hyperplasia. A neoplastic or hyperproliferative condition could be treated by a method comprising the steps of:

a) administering to the human or animal a pharmaceutically useful amount of a pharmaceutical composition comprising a substance capable of inducing apoptosis; and

b) administering to the patient a chemotherapeutic agent for the treatment of neoplasia; and/or

c) exposing the human or animal to radiation treatment.

By enhancing mammalian mitochondrial DNA gene expression in a living mammalian cell, it should also be possible to inhibit apoptosis of said mammalian cell. This could be achieved by adding a substance capable of enhancing mammalian mitochondrial DNA gene expression, and in particular affecting

a) mitochondrial DNA replication;

b) mitochondrial DNA maintenance and stability;

c) mitochondrial DNA transcription;

d) processing and stability of mitochondrial transcripts;

e) mitochondrial protein translation; and/or

f) stability of mitochondrially encoded proteins.

In particular, said enhanced gene expression could be obtained by adding a substance capable of enhancing expression of genes encoding mitochondrial RNA polymerase (SEQ. ID. NO. 2), mitochondrial transcription factor A (mtTFA or TFAM (SEQ. ID. NO. 4), mitochondrial single strand binding protein (mtSSB) (SEQ. ID. NO. 10), ribonucleotidase mitochondrial RNA processing (RNAse MRP) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), ribonucleotidase P (RNAse P) (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), the catalytic or accessory subunit of mitochondrial DNA polymerase (SEQ. ID. NO. 26, SEQ. ID. NO. 28), and mammalian homologues of yeast Mtf1, herein referred to as TFB1M (SEQ. ID. NO. 6) and TFB2M (SEQ. ID. NO. 8). Suitable compounds capable of directly affecting the function or activity of the above nuclear gene products can by found by applying the method disclosed in Example 6 below.

By inhibiting apoptosis, and thereby decreasing cell death, it should be possible to treat humans or animals having a disease or a condition characterized by increased cell death, exemplified to, but not limited to, juvenile and adult onset diabetes mellitus, Alzheimer's disease, Parkinson's disease, other neurodegenerative conditions, heart failure and the process of aging.

The present invention also relates to a method for in vitro identifying a substance capable of impairing mammalian mitochondrial DNA gene expression. Such a substance is capable of inducing apoptosis of a living mammalian cell. The method comprises the steps of:

a) providing a substance suspected of impairing mammalian mitochondrial DNA gene expression by affecting the expression of nuclear genes regulating mitochondrial DNA replication, mitochondrial DNA maintenance and stability, mitochondrial DNA transcription, the processing and stability of mitochondrial transcripts, mitochondrial protein translation or the stability of mitochondrially encoded proteins;

b) contacting the substance in step a) with a compound chosen from the group of

i) mitochondrial RNA polymerase (SEQ. ID. NO. 1) or the corresponding DNA/RNA sequence (SEQ. ID. NO. 2);

ii) mitochondrial transcription factor A (TFAM) (SEQ. ID. NO. 3)) or the corresponding DNA/RNA sequence (SEQ. ID. NO. 4);

iii) mitochondrial transcription factor B (TFB1M or TFB2M (SEQ. ID. NO. 5, SEQ. ID. NO. 7) ) or the corresponding DNA/RNA sequence (SEQ. ID. NO. 6, SEQ. ID. NO. 8);

iv) Homo sapiens ribonuclease P and RNAse MRP subunits (SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23)) or the corresponding DNA/RNA sequence (SEQ. ID. NO. 12, SEQ. ID. NO. 14, SEQ. ID.. NO. 16, SEQ. ID. NO. 18, SEQ. ID. NO. 20, SEQ. ID. NO. 22, SEQ. ID. NO. 24);

v) the catalytic or accessory subunit of mitochondrial DNA polymerase (SEQ. ID. NO. 25, SEQ. ID. NO. 27)) or the corresponding DNA/RNA sequence (SEQ. ID. NO. 26, SEQ. ID. NO. 28); and

vi) fragments of the above compounds comprising at least 15 consecutive amino acids or at least 45 consecutive nucleotides; and

c) determining whether the substance in step a) binds to the compound of step b), thereby impairing mammalian mitochondrial DNA gene expression.

Preferably, the compound in step b) is an enzyme chosen from mitochondrial RNA polymerase (SEQ. ID. NO. 1), TFAM (SEQ. ID. NO. 3), TFB1M or TFB2M (SEQ. ID. NO. 5, SEQ. ID. NO. 7), Homo sapiens ribonuclease P and RNAse MRP subunits (SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23), and mitochondrial DNA polymerase (SEQ. ID. NO. 25, SEQ. ID. NO. 27). Still more preferably, it is determined whether the substance in step a) upon contact affects the enzymatic activity of the enzyme in step b).

A compound that has been identified by the above method can be used for preparing a pharmaceutical composition for treating cancer, lymphoproliferative syndromes, autoimmune diseases, sarcomas, meningeomas, basal cell carcinomas, benign tumours, psoriasis, or prostatic hyperplasia, diabetes mellitus, heart failure, neurodegeneration, obesity or hormonal disturbances.

The enclosed sequence listing comprises the following sequences:

SEQ. ID. NO. 1: Human mitochondrial RNA polymerase, amino acid sequence;

SEQ. ID. NO. 2: Human mitochondrial RNA polymerase, cDNA sequence;

SEQ. ID. NO. 3: Homo sapiens mitochondrial transcription factor A, amino acid sequence;

SEQ. ID. NO. 4: Homo sapiens mitochondrial transcription factor A, cDNA sequence;

SEQ. ID. NO. 5: Homo sapiens TFB1M (CGI-75 protein), amino acid sequence;

SEQ. ID. NO. 6: Homo sapiens TFB1M (CGI-75 protein), cDNA sequence;

SEQ. ID. NO. 7: Homo sapiens TFB2M, partial amino acid sequence, carboxy terminal;

SEQ. ID. NO. 8: Homo sapiens TFB2M, partial cDNA, 5′-terminal;

SEQ. ID. NO. 9: Homo sapiens single-stranded DNA-binding protein (SSBP), amino acid sequence;

SEQ. ID. NO. 10: Homo sapiens single-stranded DNA-binding protein (SSBP), cDNA sequence;

SEQ. ID. NO. 11: Homo sapiens ribonuclease P and RNAse MRP subunit (14 kD) (RPP14), amino acid sequence;

SEQ. ID. NO. 12: Homo sapiens ribonuclease P and RNAse MRP subunit (14 kD) (RPP14), cDNA sequence;

SEQ. ID. NO. 13: Homo sapiens ribonuclease P and RNAse MRP subunit p20 (RPP20) amino acid sequence;

SEQ. ID. NO. 14: Homo sapiens ribonuclease P and RNAse MRP subunit p20 (RPP20), cDNA sequence;

SEQ. ID. NO. 15: Homo sapiens ribonuclease P and RNAse MRP subunit p29 (RPP29), amino acid sequence;

SEQ. ID. NO. 16: Homo sapiens ribonuclease P and RNAse MRP subunit p29 (RPP29), cDNA sequence;

SEQ. ID. NO. 17: Homo sapiens ribonuclease P and RNAse MRP subunit (RPP30), amino acid sequence;

SEQ. ID. NO. 18: Homo sapiens ribonuclease P and RNAse MRP subunit (RPP30), cDNA sequence;

SEQ. ID. NO. 19: Homo sapiens ribonuclease P and RNAse MRP subunit (RPP38), amino acid sequence;

SEQ. ID. NO. 20: Homo sapiens ribonuclease P and RNAse MRP subunit (RPP38), cDNA sequence;

SEQ. ID. NO. 21: Homo sapiens ribonuclease P and RNAse MRP subunit (RPP40), amino acid sequence;

SEQ. ID. NO. 22: Homo sapiens ribonuclease P and RNAse MRP subunit (RPP40), cDNA sequence;

SEQ. ID. NO. 23: Homo sapiens homolog to Saccharomyces cerevisiae ribonuclease P and RNAse MRP subunit Pop1, or human KIAA0061, amino acid sequence;

SEQ. ID. NO. 24: Homo sapiens homolog to Saccharomyces cerevisiae ribonuclease P and RNAse MRP subunit Pop1, or human KIAA0061,cDNA sequence;

SEQ. ID. NO. 25: Homo sapiens polymerase (DNA directed), gamma (POLG), nuclear gene encoding mitochondrial protein, amino acid sequence;

SEQ. ID. NO. 26: Homo sapiens polymerase (DNA directed), gamma (POLG), nuclear gene encoding mitochondrial protein, cDNA sequence;

SEQ. ID. NO. 27: Homo sapiens polymerase (DNA directed), gamma 2, accessory subunit (POLG2), amino acid sequence;

SEQ. ID. NO. 28: Homo sapiens polymerase (DNA directed), gamma 2, accessory subunit (POLG2), cDNA sequence;

### General Discussion Regarding Experimental Results

Respiratory chain dysfunction contributes to human pathology by affecting cellular energy production and can produce symptoms from almost any organ with almost any age of onset. Cell loss has been documented in the brain stem and pancreatic islets in humans with deficient respiratory chain function. We have recently documented loss of β-cells in mice with β-cell-specific disruption of Tfam [Silva et al., Nature Genet. 26:336-340 (2000)]. It is thus clear that deficient respiratory chain function may cause cell loss in vivo, but the cell loss mechanism has remained elusive.

In this paper we document apoptotic cell death in mouse embryos and mouse hearts with respiratory chain deficiency. In both cases we found significant increase of TUNEL positive cells indicative of an active apoptotic process. We further confirmed apoptosis in Tfam knockout hearts by showing DNA fragmentation with gel electrophoresis. We also detected activated caspase 3 in Tfam knockout mouse embryos and activated caspase 3 and activated caspase 7 in Tfam knockout hearts. The respiratory chain deficiency cause a major mutant phenotype [Larsson et al., Nature Genet. 18:231-236 (1998)] in the Tfam knockout embryos without increased apoptosis at E8.5, followed by a massive apoptosis at E9.5 and resorption of the embryo at E10.5. Our findings show that both embryonic and differentiated cells lacking mtDNA can undergo apoptosis in vivo. It is thus possible that apoptosis may contribute significantly to the pathology observed in patients with mtDNA mutation disorders. However, the limited supply of human tissues has been a major drawback to study this phenomenon in humans and we are only aware of a single report indicating increased apoptosis in human mtDNA mutation disorders [Mirabella et al., Brain 123:93-104 (2000)].

Recent studies have reported that mtDNA-depleted osteosarcoma cells [Dey et al., J. Biol. Chem. 275:7087-7094 (2000)] or mouse embryonic cytochrome c knockout cells [Li et al., Cell 101:389-399 (2000)] are less susceptible to apoptosis induction by staurosporin (STP) and serum depletion, raising the possibility that respiratory chain function is important for executing apoptosis. We therefore reinvestigated the apoptotic phenotype of cells lacking mtDNA. Our data show that mtDNA-depleted osteosarcoma cells can undergo apoptosis in vitro in response to a variety of signals, i.e. STP and death receptor activation. These findings are in agreement with previous studies carried out on other cell lines lacking mtDNA [Jacobson et al., Nature 361:365-369 (1993); Jiang et al., J. Biol. Chem. 274:29905-29911 (1999); Marchetti et al., Cancer Res. 56:2033-2038 (1996)].

The cell death mechanism, apoptosis or necrosis, has been shown to depend on intracellular ATP levels [Leist, et al., J. Exp. Med. 185:1481-1486 (1997)]. ATP depletion blocks nuclear condensation and DNA fragmentation in the final phase of STP- and Fas-induced apoptosis of human T cells [Leist et al., J. Exp. Med. 185:1481-1486 (1997)]. We could not detect inflammatory or post-inflammatory signs, expected to result from necrosis, in Tfam knockout hearts. This result suggests that there is sufficient ATP supply to allow cardiomyocytes to undergo apoptosis despite the impaired oxidative phosphorylation. Consistent with this hypothesis, we found increased Gapdh transcript levels indicative of a compensatory upregulation of glycolysis.

Previous studies have characterized the molecular events involved in the apoptotic response of cell lines with respiratory chain deficiency. When U937 cells lacking mtDNA undergo apoptosis in response to TNFα plus cycloheximide, there is initially decreased mitochondrial membrane potential and increased ROS formation later followed by DNA fragmentation [Marchetti et al., Cancer Res. 56:2033-2038 (1996)]. Furthermore, mitochondria isolated from mtDNA depleted U937 cells can undergo permeability transition with release of apoptogenic factors [Marchetti et al,, Cancer Res. 56:2033-2038 (1996)]. These results suggest that ρ⁰ cells are able to induce the mitochondrial pathway for apoptosis. This has been furtherer corroborated by studies of mtDNA-depleted osteosarcoma cells demonstrating that cytochrome c-mediated apoptosis is conserved in these cells [Jiang et al., J. Biol. Chem. 274:2990S-29911 (1999)]. However, in vitro studies depend on mutant cell lines which are aneuploid and considerable differences of the karyotype are present in ρ⁺ and ρ⁰ cell lines [Hao et al., Hum. Mol. Genet. 8:1117-1124 (1999)]. It is thus impossible to conclude from these studies that only the respiratory chain dysfunction influences the susceptibility of different apoptotic pathways. It therefore remains open if cytochrome c-mediated apoptosis is the main in vivo pathway in cells lacking mtDNA or if other, cytochrome c-independent pathways may contribute to the apoptotic response. The methods to study apoptotic pathways in vivo are of limited power and repeated attempts to establish Tfam knockout cell lines for in vitro studies have so far failed (unpublished data). However, our data provide the first genetic evidence that respiratory chain deficient cells are predisposed to undergo apoptosis in vivo.

The finding that respiratory chain deficiency is associated with increased in vivo apoptosis may have important therapeutic implications for human disease. Respiratory chain dysfunction has been suggested to be of pathophysiological importance in a wide variety of common diseases, e.g neurodegeneration, heart failure and diabetes mellitus, and aging. Interestingly, cell loss and/or apoptosis have been described in all of these conditions. Impaired apoptosis is suggested to be of importance for the development of malignant tumors and various hyperproliferative syndromes. Furthermore, chemotherapy and radiation treatment of cancer aims at inducing apoptosis in the tumor cells. It is thus possible that manipulation of respiratory chain function may be utilized to enhance or inhibit apoptosis in a wide variety of conditions.

The present invention will now be further described with references to the enclosed figures, in which:

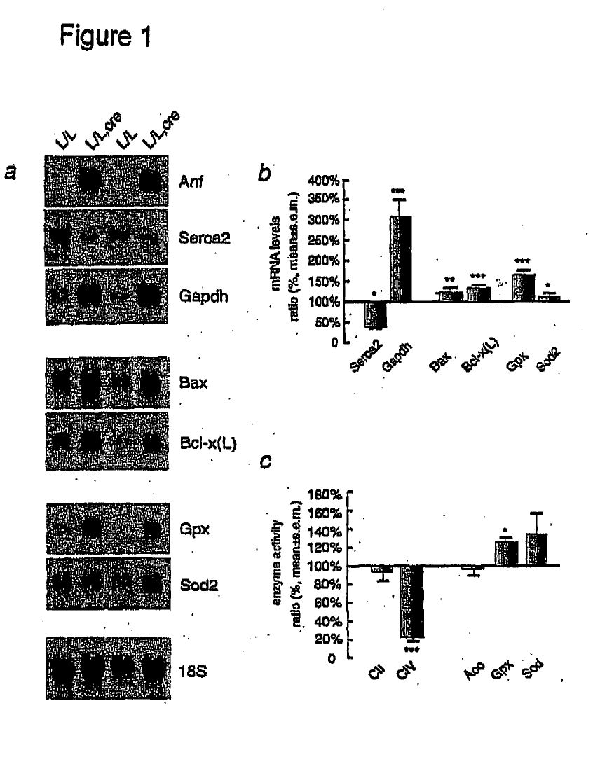

FIG. 1 shows gene expression profiles and mitochondrial enzyme activities in hearts of Tfam heart knockouts (Tfam^{loxP}/Tfam^{loxP}, +/Ckmm-cre) and littermate controls (Tfam^{loxP}/Tfam^{loxP}). (a), Northern blots showing mRNA expression of atrial natriuretic factor (Anf), cardiac sarcoplasmic reticulum Ca²⁺ ATPase2 (Serca2), glyceraldehyde-3-phosphate dehydrogenase (Gapdh), Bax, Bcl-x(L), glutathione peroxidase (Gpx), and mitochondrial superoxide dismutase (Sod2) in Tfam knockout hearts (L/L, cre) and control hearts (L/L), The nuclear 18S rRNA transcript was used as a loading control. (b), Results from phosphoimager analyses of gene transcript levels in Tfam knockout (n=4) and control hearts (n=4). The relative transcript levels in Tfam knockout hearts in comparison with control hearts are shown. *, P<0.05; **, P<0.01; ***, P<0.001. (c), Biochemical measurements of complex II (CII) and complex IV (CIV), aconitase (Aco), glutathione peroxidase (Gpx), and total superoxide dismutase (Sod) activities in Tfam knockout (n=8) and control hearts (n=8). The relative enzyme activities in Tfam knockout hearts in comparison with control hearts are shown. *, P<0.05; ***, P<0.001;

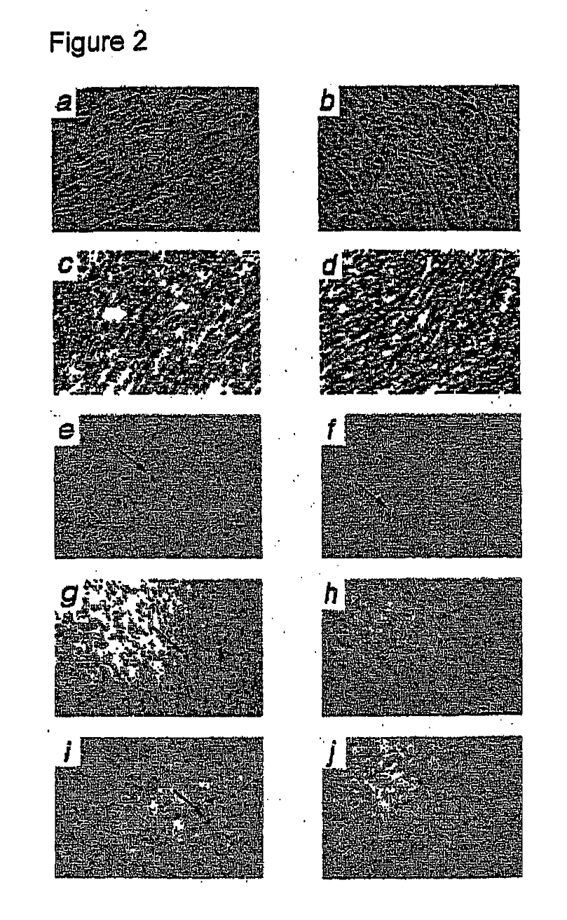

FIG. 2 discloses histology of hearts from Tfam heart knockouts (Tfam^{loxP}/Tfam^{loxP}, +/Ckmm-cre) and their littermate controls (Tfam^{loxP}/)Tfam^{loxP}). Examples of immunoreactive cells are indicated by arrows. Trichrome stainings show no evidence for necrosis or fibrosis in Tfam knockout (a) or control (b) hearts. Double enzyme histochemical stainings for cytochrome c oxidase (COX) activity and succinate dehydrogenase (SDH) activity show a mosaic loss of COX activity in Tfam knockout hearts as evidenced by the blue staining of cardiomyocytes (c) and normal COX activity in controls as reflected by the brown staining of cardiomyocytes (d). TUNEL stainings demonstrate more TUNEL positive cardiomyocytes in Tfam knockout hearts (e) than in control hearts (f). Immunohistochemical stainings of cleaved caspase 3 and cleaved caspase 7 show occasional positive cardiomyocytes in Tfam knockout hearts (g, i) and no staining in control hearts (h, j);

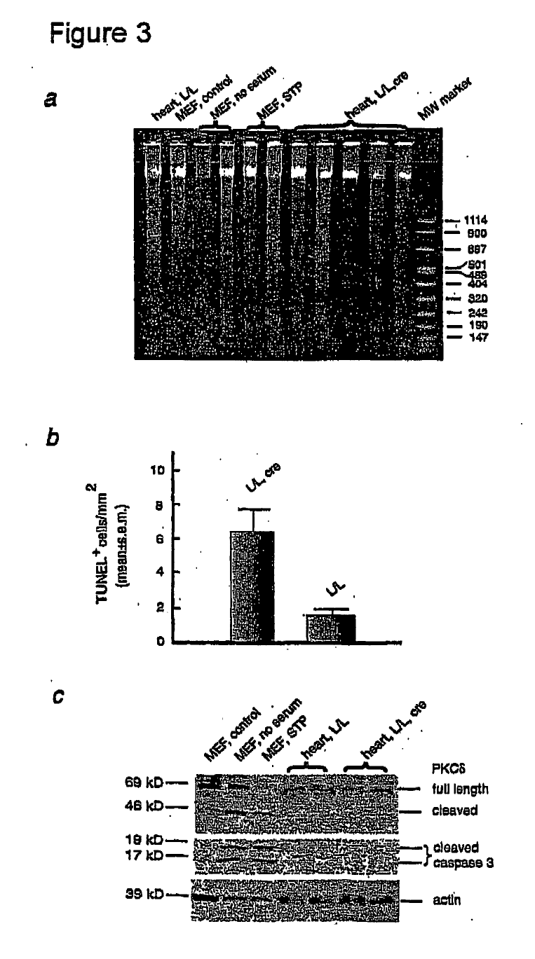

FIG. 3 shows that Tfam knockout hearts (Tfam^{loxP}/Tfam^{loxP}, +/Ckmm-cre) show increased apoptosis. (a), DNA ladders can be detected in Tfam knockout hearts (heart, L/L, cre) but not in control hearts (L/L). Serum starved (no serum) and staurosporine treated (STP) mouse embryonic fibroblasts (MEF) were used as positive controls, untreated MEF (MEF, control) were used as negative controls. (b), Wilcoxon matched pairs test of results from TUNEL stainings of Tfam knockout hearts (n=15) and control hearts (n=15). Values represent number of TUNEL positive cells/mm² section. *, P<0.001. (c), Detection of caspase 3 and PKCδ cleavage by Western blot analysis. Cleaved caspase 3 and cleaved PKCδ were not detectable in Tfam knockout hearts (L/L, cre) and control hearts (L/L). Serum-starved (MEF, no serum) and STP-treated MEF (MEF, STP) were used as positive controls and untreated MEF (MEF, control) as negative controls;

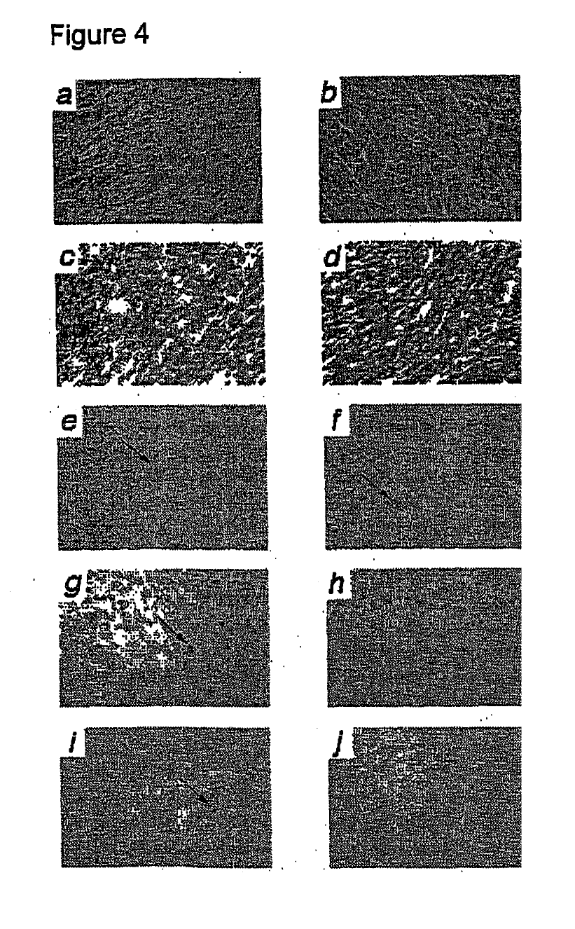

FIG. 4 discloses that massive apoptosis occur in embryonic day (E) 9.5 Tfam knockout (Tfam^{-/-}) embryos. All panels illustrate results from embryos at E9.5. Enzyme histochemical staining for cytochrome c oxidase (COX) activity shows no COX activity in Tfam knockout embryos (a) and normal COX activity in control embryos (b). Enzyme histochemical stainings for succinate dehydrogenase (SDH) activity were normal in Tfam knockout (c) and control embryos (a). TUNEL staining demonstrates abundant TUNEL positive cells (arrows) in Tfam knockout embryos (e) and few positive cells in control embryos (f). Immunohistochemical stainings to detect cleaved caspase 3 show abundant positive cells (arrows) in Tfam knockout embryos (g) and occasional positive cells in control embryos (h). Immunohistochemical stainings to detect cleaved caspase 7 are negative in Tfam knockout (i) and control embryos (f); and

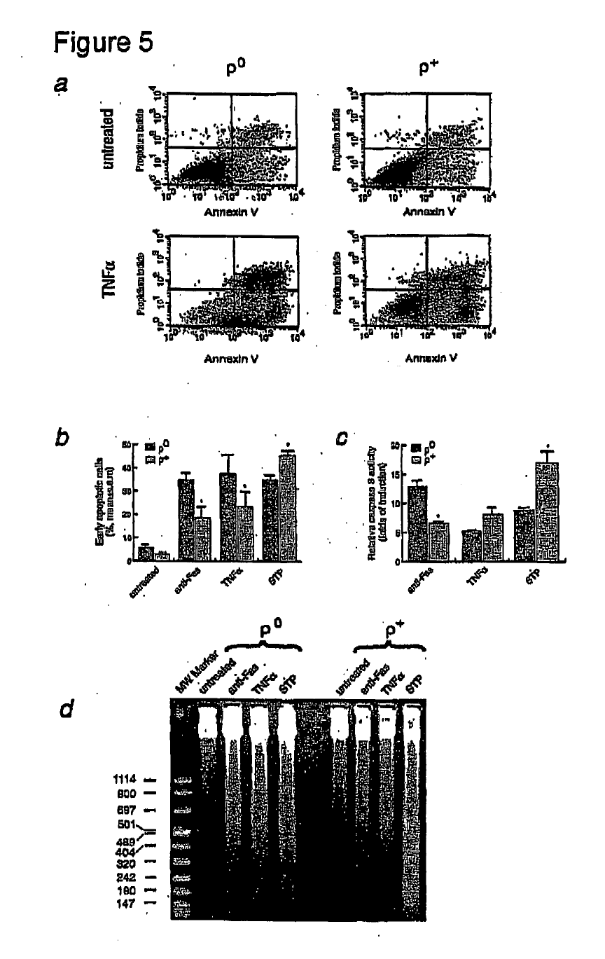

FIG. 5 shows that ρ⁰ cells are susceptible to apoptosis induced by various signals. ρ⁰ (143B/206) and ρ⁺ (143B) osteosarcoma cells were incubated for 16 hours with 0.5 μM staurosporine (STP), 100 ng/ml anti-Fas antibody plus 100 ng/ml actinomycin D (anti-Fas), or 20 ng/ml TNFα plus 100 ng/ml actinomycin D (TNFα). (a), analysis by flow cytometry of apoptotic cells stained with annexin V (Ax) and propidium iodide (PI) to distinguish early apoptotic cells (Ax positive, PI negative) from late apoptotic or necrotic cells (Ax positive, PI positive). (b), Susceptibility of ρ⁰ (143B/206) and ρ⁺ (143B) osteosarcoma cells to undergo apoptosis in response to various signals as determined by annexin V/propidium iodide staining and flow cytometry. Values represent the percentage of early apoptotic (annexin V positive/propidium iodide negative) cells (%). *, P<0.05; **, P<0.01. (c) Caspase 3 activities in ρ⁰ (143B/206) and ρ⁺ (143B) osteosarcoma cells. The results are plotted as fold induction of caspase 3 activity compared to untreated cells. *, P<0.05. (d), DNA ladders in ρ⁰ (143B/206) and ρ⁺ (143B) osteosarcoma cells.

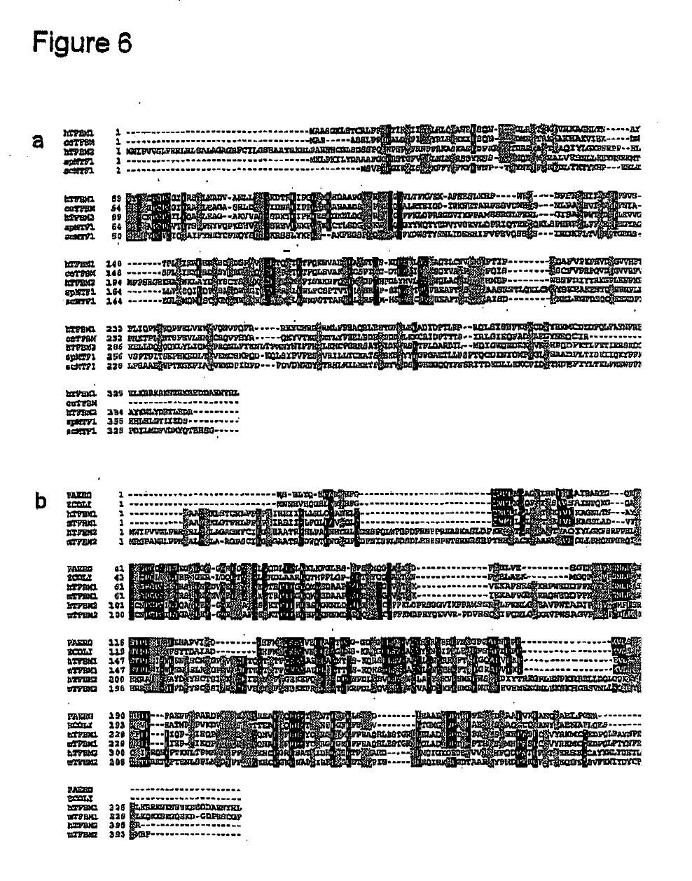

FIG. 6 presents alignment of the predicted amino acid sequences of mitochondrial transcription factor B (TFBM) homologoues. (a) The sequences for human TFB1M (hTFB1M, NP_{—}057104), human TFB2M (hTFB2M, NP_{—}071761), Caenorhabditis elegans TFBM (ceTFBM, T29195), Schizosaccharomyces pombe Mtf1 (spMtf1, CAB65608) and Saccharomyces cerevisiae Mtf1 (scMtf1, NP_{—}013955) are shown. Regions with sequence identity or similarity greater than 75% are shaded. The TFB2M sequence exhibited 25% identity and 45% similarity (E=5×10⁻⁷) to a region spanning amino acids 61 and 217 in TFB1M. (b) Mouse and human TFB1M display strong sequence similarity to bacterial RNA dimethylases. The sequences for Pseudomonas aeruginosa dimethyladenosine transferase (PAERG, H83571), Eschericha coli (ECOLI, P06992) dimethyladenosine transferase, human TFB1M, mouse TFB1M (mTFB1M, cDNA sequenced by us, seq. id. not yet obtained.), human TFB2M, and mouse TFB2M (mTFB2M, NP_{—}032275) are shown. Regions with sequence identity or similarity greater than 65% are shaded.

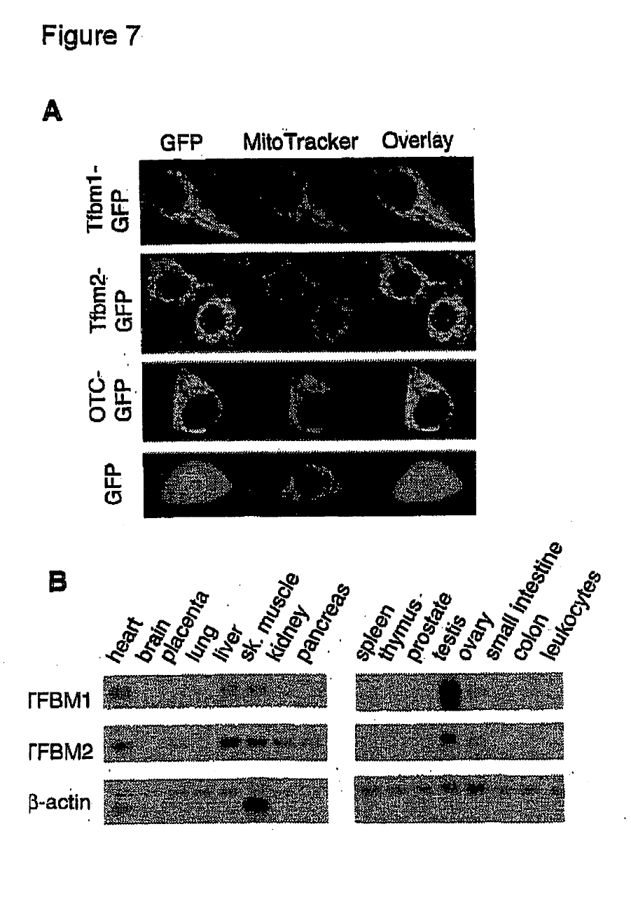

FIG. 7 shows subcellular localization and expression of TFB1M and TFB2M. (A) Confocal microscopy images of human cells transfected with plasmids encoding GFP-tagged mouse Tfb1m (Tfb1m-GFP), GFP-tagged mouse Tfb2m (Tfb2m-GFP), mitochondrially targeted GFP (OTC-GFP) and non-targeted GFP (GFP). MitoTracker specifically stains mitochondria. (B) Northern blot analysis of the expression of TFB1M and TFB2M in different human tissues. A single TFB1M transcript of ˜1.3 kb and a single TFB2M transcript of ˜1.7 kb is present in all investigated tissues. A β-actin loading control is also shown.

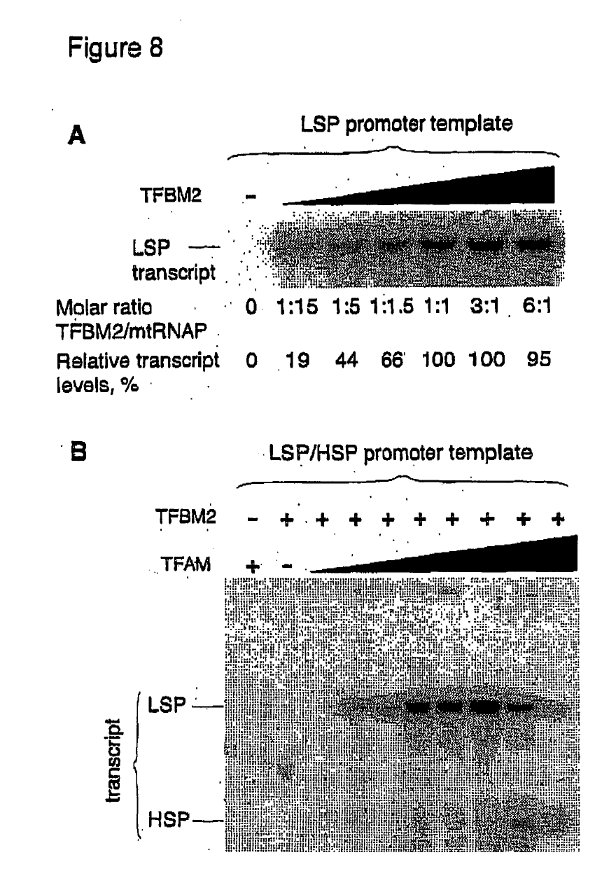

FIG. 8 relates to characterization of mitochondrial in vitro transcription: (A) SDS-PAGE gel stained with Coomassie blue depicting the different recombinant human proteins used for in vitro transcription reactions. (B) In the presence of TFAM (2.5 pmol), mtRNAP/TFB1M (400 fmol) or mtRNAP/TFB2M (400 fmol) can support transcription in vitro. The transcriptional activation obtained with TFB2M is at least one order of magnitude greater than the activation obtained with TFB1M. (C) A transcription system containing TFAM (2.5 pmol), mtRNAP/TFB2M (400 fmol) can support transcription from both LSP and HSP. (D). Transcription from LSP only occurs when TFAM (2.5 pmol), mtRNAP (400 fmol), and TFB2M (400 fmol) are present simultaneously.

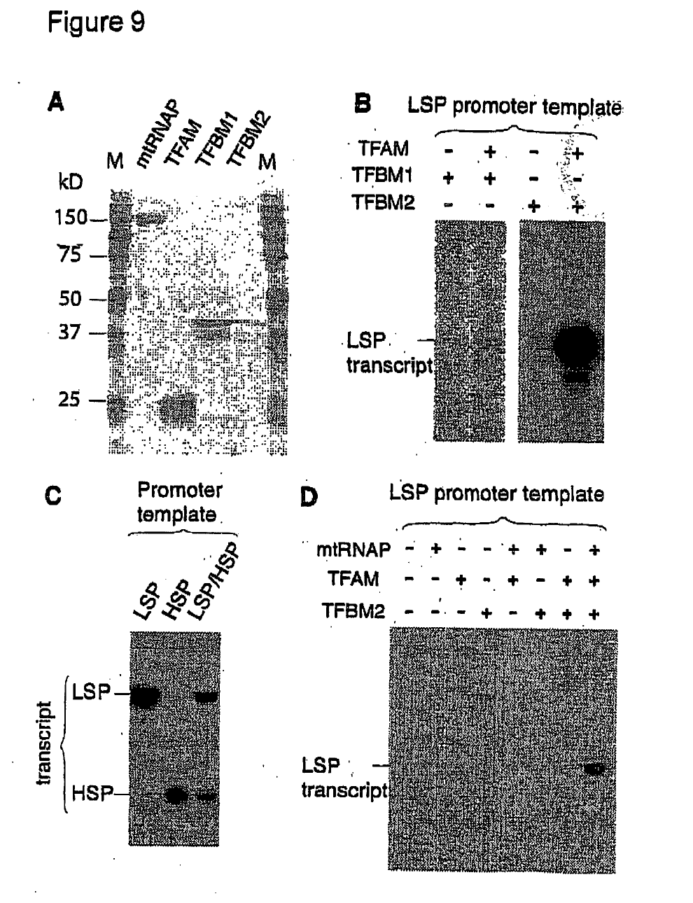

FIG. 9 presents effects of TFB2M ad TFAM concentrations on mitochondrial transcription in vitro. (A). Maximal transcription activity occurs at a 1:1 molar ratio of TFB2M and mtRNAP. The in vitro transcription reaction mixtures contained 1.3 pmol of TFAM, 250 fmol of mtRNAP and 85 fmol of LSP-template. Increasing amounts of TFB2M were added as indicated. The molar ratio of TFB2M to mtRNAP and the relative levels of transcription are shown. (B). The concentration of TFAM required for transcription from LSP and HSP differs. The reaction mixture contained 400 fmol of mtRNAP, 400 fmol TFB2M, and 85 fmol of LSP/HSP-template. Increasing amounts of TFAM (0.025, 0.075, 0.25, 0.75, 2.5, 7.5, 15 and 22.5 pmol) were added.

### EXPERIMENTAL PART

### Materials and Methods

Tissue Samples

Mice with heart-specific disruption of Tfam were generated as described [Wang et al, Nature Genet. 21:133-131 (1999)]. Heart samples from Tfam heart knockouts (Tfam^{loxP}/Tfam^{loxP}, +/Ckmm-cre) and their littermate controls (Tfam^{loxP}/Tfam^{loxP}) were collected at around 2-3 weeks of age. Homozygous Tfam knockout embryos (Tfam^{-/-} were obtained by matings between germline heterozygous Tfam knockout animals (Tfam^{+/-}) [Larsson et al., Nature Genet. 18:231-236 (1998)]. Pregnant females were sacrificed at 8.5 or 9.5 days post coitum and decidua containing embryos were collected. The samples were immediately embeaded in O.C.T™ Tissue-Tek (Sakura, The Netherlands) and kept at −70° C. until further use.

Cell Lines

A human osteosarcoma-derived cell line, 143B, containing mtDNA (ρ⁺), and its mtDNA-less derivative, 143B/206 (ρ⁰), were maintained in Dulbecco's Modified Eagle Medium (DMEM)-high glucose (1000 MG/L; GibcoBRL, Life Technologies AB, Sweden) containing 10% fetal bovine serum, and 100 IU/ml penicillin-streptomycin (GibcoBRL, Life Technologies AB, Sweden). The 143B/206 ρ⁰ cells were additionally supplemented with 1 mM sodium pyruvate (GibcoBRL, Life Technologies AB, Sweden) and 50 μg/ml uridine (Sigma-Aldrich AB, Sweden) as described [King et al, Science 246:500-503 (1989)]. Cells were grown to sub-confluence.

Cytotoxicity Assays

Cells were incubated for 16 hours at 37° C. with medium containing: 1) 0.5 μM staurosporine (Sigma-Aldrich AB, Sweden); 2) 100 ng/ml human anti-Fas antibody (MBL, Nagoya, Japan) plus 100 ng/ml actinomycin D (Sigma-Aldrich AB, Sweden); 3) 20 ng/ml human recombinant tumour necrosis factor α (TNFα, Upstate Biotechnology, USA) plus 100 ng/ml actinomycin D. Cells were pretreated with 100 ng/ml actinomycin D for 15 minutes at 37° C. prior to addition of TNFα plus actinomycin D or anti-Fas antibody plus actinomycin D.

Flow Cytometric Analyses of Apoptotic Cells

We stained the cells with annexin V and propidium iodide using the Vybrant apoptosis assay kit 2 (Molecular Probes, Leiden, The Netherlands). Flow cytometric analyses were performed on a Beckton Dickinson flow cytometer (FACScan) and the results were analyzed by using the Cell Quest program (Beckton Dickinson). Annexin V/propidium iodide measurements were performed on ρ⁰ and ρ⁺ cells incubated with 0.5 μM staurosporine (n=3), 100 ng/ml human anti-Fas antibody plus 100 ng/ml actinomycin D (n=4), and 20 ng/ml human recombinant tumour necrosis factor α plus 100 ng/ml actinomycin D (n=4) for 16 hours.

TUNEL Assay

Cryostat tissue sections of hearts or embryos and slides with tissue-culture cells were fixed in 1% paraformaldehyde in phosphate buffered saline for 10 minutes at room temperature. TUNEL staining was carried out using the Apoptag Peroxidase Kit (Invitrogen, USA). Sections were counterstained with Methyl Green (DAKO, Carpinteria, Calif.). Areas of heart sections were measured with the NIH Image 1.41 program (http://rsb.info.nih.gov/nih-image). TUNEL positive cells on the whole section were counted, and the apoptotic index was calculated as the number of TUNEL positive cells/mm². TUNEL stainings were performed on heart sections from 2-3 week old Tfam heart knockouts (n=15) and littermate controls (n=15) and from Tfam knockout embryos (Tfam^{-/-}) and littermate control embryos at E8.5 (n=3) and E9.5 (n=4).

DNA Ladder Assay

Tissues and cells were incubated for 3 hours at 50° C. in lysis buffer (50 mM Tris-HCl (pH 8.0), 0.1M NaCl, 2.5 mM EDTA, 0.5% SDS and 200 μg/ml proteinase K). DNA was isolated with chloroform extraction and treated with 1 μg/ml DNase-free RNase (Boehringer Mannheim Scandinavia, Sweden) for one hour at room temperature. DNA samples (10-20 μg) were separated by electrophoresis in a 1.8% agarose gel. The gel was stained with SYBR Green I nucleic acid gel stain (Molecular Probes, Leiden, The Netherlands) after electrophoresis and the DNA was visualised under UV light.

Measurement of Caspase 3 Activity

Caspase 3 activity was measured by the caspase 3 assay kit (Pharmingen, CA, USA). Briefly, a tetrapeptide labeled with the fluorochrome 7-amino-4-methylcoumarin (AMC) was used as a substrate to identify and quantitate caspase 3 activity. AMC is released from the substrate upon cleavage by caspase-3. Free AMC is quantified in cell lysates by ultraviolet (UV) using an excitation wavelength of 365 nm and an emission wavelength of 460 nm. The fluorometric count was normalized by the protein concentration of the supernatant. Caspase 3 activity was measured on ρ⁰ and ρ⁺ cells incubated with 0.5 μM staurosporin (n=3), 100 ng/ml human anti-Fas antibody plus 100 ng/ml actinomycin D (n=3), and 20 ng/ml human recombinant tumour necrosis factor α plus 100 ng/ml actinomycin D (n=3) for 16 hours.

Northern Blot

RNA from heart samples was isolated with the Trizol Reagent (GibcoBRL, Life Technologies AB, Sweden). RT-PCR products were separated on gels, purified with the QIAEX II gel extraction kit (Qiagen, Germany), radiolabelled with α³²P and used as probes to detect glyceraldehyde-3-phosphate dehydrogenase (Gapdh), atrial natriuretic factor (Anf), sarcoplasmic reticulum Ca²⁺ ATPase2 (Serca2), Bcl-x(L), Bax, glutathione peroxidase (Gpx) and mitochondrial superoxide dismutase (Sod2) transcripts. The intensity of signals were recorded by a Fujix Bio-Imaging Analyzer BAS 1000 (FujiFilm) and data were analysed with Image Gauge V3.3 program (FujiFilm). The Loading was normalized to 18S rRNA.

Histology and Biochemistry

Cryostat tissue sections from hearts or embryos and slides with tissue-culture cells were fixed for 10 minutes at room temperature in phosphate-buffered 1% paraformaldehyde followed by permeabilization in ice-cold acetic acid/ethanol for 5 minutes. We used polyclonal antisera against: 1) cleaved caspase 3 (Cell signalling technology, New England Biolabs, USA); 2) cleaved caspase 7 (Cell signalling technology, New England Biolabs, USA); 3) p53 (Santa Cruz Biotechnology, USA). We incubated the sections with primary antibodies at 4° C. for overnight at the recommended dilutions and used Dako Envision™ (Dako, USA) as a secondary antibody. Immunohistochemical stainings to detect cleaved caspase 3 and 7 were performed on heart sections from Tfam heart knockouts (n=4-7) and littermate controls (n=4-7) and from E9.5 Tfam knockout (n=4) and control embryos (n=4). Immunohistochemical stainings to detect p53 were carried out on heart sections from Tfam heart knockouts (n=3) and littermate controls (n=3). Enzyme histochemical stainings to detect cytochrome c oxidase (COX) and succinate dehydrogenase (SDH) activity were performed on cryostat sections as described [Larsson et al., Nature Genet. 18:231-236 (1998)]. Biochemical measurements of enzyme activities were performed on hearts from Tfam heart knockouts (n=8) and littermate controls (n=8) as described [Rotig et al., Nature Genet. 17:215-217 (1997); Rustin et al., Clin. Chimica Acta 228:35-51 (1994)].

Western Blots

Total protein extracts were prepared from tissue samples and cultured cells as described [Wang et al., Nature Genet. 21:133-137 (1999)]. Total protein (50-100 μg) was separated in a 10-20% polyacrylamide gradient gel (Bio-Rad Laboratories AB, Sweden) and blotted to Hybond™-C extra membranes (Amersham Life Science). Membranes were blocked in 5% non-fat milk and then incubated with the primary antibodies at 4° C. for overnight at recommended dilutions. The primary antibodies reacted with p53 (Santa Cruz Biotechnology, USA), cleaved caspase 3 (Cell signalling technology, New England Biolabs, USA), and PKCδ (Santa Cruz Biotechnology, USA). We used horseradish peroxidase (HRP)-conjugated goat anti-rabbit Ig (1:2000) (Amersham Life Science) as secondary antibody. The signal was detected by enhanced chemiluminescence (Amersham Life Science).

Expression and Purification of Recombinant Proteins

Genes encoding TFB1M, TFB2M, mtRNAP, and TFAM were PCR amplified from cDNAs and cloned into the pBacPAK9 vector (Clontech). Plasmid constructs were also made in which a 10×His-tag had been introduced at the amino terminus (mtRNAP) or a 6×His-tag had been introduced at the carboxy terminus TFAM, TFB1M, TFB2M). Autographa californica nuclear polyhedrosis viruses recombinant for the individual proteins were prepared as described in the BacPAK™ manual (Clontech).

### Example 1

### Cardiomyocytes with Impaired Oxidative Phosphorylation Are More Prone to Undergo Apoptosis Than Normal Cardiomyocytes

We performed additional studies of the previously characterized transgenic mouse model with tissue-specific Tfam gene disruption causing postnatal onset of severe mitochondrial cardiomyopathy [Wang et al., Nature Genet. 21:133-137 (1999)]. Northern blots demonstrated increased levels of the transcripts for the glycolytic enzyme glyceraldehyde-3-phosphate dehydrogenase (Gapdh) and atrial natriuretic factor (Anf) (FIGS. 1a and b). Levels of sarcoplasmic reticulum Ca²⁺ ATPase2 (Serca2) transcripts were reduced (FIGS. 1a and b). These changes in gene expression are typically found in animals and humans with heart failure 12,13 [Wankerl et al., J. Mol. Med. 73:487-496 (1995); Arai et al., Circ. Res. 74:555-564 (1994)]. Histological analyses of Tfam knockout hearts showed no evidence for fibrosis, necrosis or inflammatory cell infiltration (FIG. 2a). Terminal deoxynucleotidyl transferase-mediated dUTP nick end labeling (TUNEL) staining of heart sections demonstrated a significantly increased frequency of TUNEL positive cells in the Tfam knockout hearts (FIGS. 2e and 3b). The TUNEL assay is not considered to be specific for apoptosis 14 and we performed DNA ladder gel assays, which showed significant DNA fragmentation in 5 of 12 investigated Tfam knockout hearts (FIG. 3a). Immunohistochemical analyses detected cardiomyocytes expressing activated caspase 3 and 7 in the Tfam knockout hearts (FIGS. 2g and i) but not in control hearts (FIGS. 2h and j). Western blot analysis could detect cleavage of caspase 3 and PKCδ, a substrate of active caspase 3, in serum starved or STP-treated mouse embryonic fibroblasts (MEF), but not in the Tfam knockout hearts (FIG. 3c), likely due to the significantly smaller sensitivity of the method compared with immunohistochemical detection. Northern blots of RNA from Tfam knockout hearts showed increased levels of transcripts encoding the proapoptotic Bax and the anti-apoptotic Bcl-x(L) proteins (FIGS. 1a and b), demonstrating increased expression of genes regulating apoptosis. Taken together these findings are consistent with increased apoptosis in the Tfam knockout hearts but do not provide information about the activated pathway.

### Example 2

### Germline Homozygous Tfam Knockouts Show Massive Apoptosis at Embryonic Day (E)9.5

We have previously disrupted the gene encoding Tfam in the mouse germline [Larsson et al., Nature Genet. 18:231-236 (1998)] and the resulting Tfam knockout embryos die between E8.5 and 10.5. These Tfam knockout embryos have undetectable levels of mtDNA, no functional respiratory chain and morphologically abnormal mitochondria at E8.5. Only resorbed pregnancies are recovered at E10.5 [Larsson et al., Nature Genet. 18:231-236 (1998)]. Further examination of the Tfam knockout embryos showed no increased frequency of TUNEL positive cells at E8.5 (not shown). However, at E9.5 the Tfam knockout embryos showed abundant TUNEL positive cells (FIG. 4e) and immunohistochemical staining showed increased expression of activated caspase 3 (FIG. 4g). These findings demonstrate that massive in vivo apoptosis occur in respiratory chain deficient mouse cells lacking mtDNA.

### Example 3

### ρ⁰ Cells Are Susceptible to Apoptosis Induced by Various Signals

The finding of increased apoptosis in vivo in mouse cells with a severe respiratory chain deficiency apparently contrasted with reports by others showing that human cell lines lacking mtDNA were resistant to staurosporine (STP)-induced apoptosis [Dey et al., J. Biol. Chem. 275:7087-7094 (2000)]. We therefore reinvestigated this issue in human 143B osteosarcoma cells lacking mtDNA. We used flow cytometry of cells stained with annexin V and propidium iodide to determine the number of early apoptotic cells (FIG. 5a). We found more STP-induced apoptosis in cells with mtDNA (ρ⁺ cells) than in their mtDNA-less derivatives (ρ0 cells; FIG. 5b), consistent with previous reports [Dey et al., J. Biol. Chem. 275:7087-7094 (2000)]. We further investigated death receptor pathways. Anti-Fas antibody or TNFα had no proapoptotic effect on ρ⁰ or ρ⁺ cells. We therefore sensitized the cells with actinomycin D for Fas and TNFα mediated apoptosis, as previously described [Leist et al., J. Immunol. 153:1778-1788 (1994); Latta et al., J. Exp. Med. 191:1975-1985 (2000)]. Anti-Fas antibody plus actinomycin D and TNFα plus actinomycin D induced more apoptosis in ρ⁰ cells than in ρ⁺ cells (FIGS. 5a and b). Incubation with actinomycin D had a proapoptotic effect on both ρ⁰ and ρ⁺ cells but there were no significant differences in the fraction of apoptotic cells (not shown). We measured caspase 3 activities in ρ⁰ and ρ⁺ cells treated with STP, anti-Fas antibody plus actinomycin D and TNFα plus actinomycin D and found significant induction of caspase 3 activity in both ρ⁰ and ρ⁺ cells (FIG. 5c). Activation of caspase 3 in response to these stimuli was further confirmed by Western blots and immunocytochemical stainings of ρ⁰ and ρ⁺ cells to detect the active subunits of caspase 3 (not shown). We further demonstrated the presence of DNA ladders in ρ⁰ and ρ⁺ cells treated with the same stimuli (FIG. 5d).

### Example 4

### Downregulation of mtDNA Gene Expression Cause Tumor Cell Death in Vivo

We have performed genetic experiments verifying that downregulation of mtDNA gene expression makes tumor cells more responsive to cell death induced by treatment with chemotherapy and radiation. Furthermore, tumor cells with downregulation of mtDNA gene expression are less prone to metastasis. These results provide the intellectual and experimental framework establishing that development of drugs interfering with mtDNA gene expression will be a valuable treatment for neoplasia and hyperproliferative syndromes.

We harvested mouse embryos of the genotype Tfam^{loxP}/Tfam^{loxP} (Larsson et al, Nature Genetics 1998:18:231-236) and established mouse embryonic fibroblast (MEF) cell cultures by using standard protcols (Hogan, Beddington, Constantini, Lacy. Manipulating the mouse embryo—A laboratory manual, Cold Spring Harbor Laboratory Press, 1994). We used standard protocols (Meek et al., Exp. Cell Res. 1977:107:277-284, Todaro and Green, J. Cell Biol. 111963:17:299-313) to transform MEF primary culture cells and immortal cell lines were established. Cell lines were transfected with constructs containing inducible promoters controlling the expression of the Tfam cDNA and the endogenous Tfam gene was disrupted by transient cre-expression.

The resulting cell lines, containing a homozygous knockout for the endogenous Tfam gene and an introduced regulatable Tfam transgene, were further investigated. We found a clear correlation between Tfam protein expression and mtDNA levels and between Tfam protein expression and mtDNA transcript levels. We found that cell lines with low Tfam protein expression were more sensitive to in vitro apoptosis induction by a variety of agents. We also implanted the cell lines with regulatable Tfam transgenes subcutaneously in nude mice. These in vivo experiments revealed that tumors with high Tfam protein expression were more prone to metastasis and more resistant to chemotherapy and radiation treatment than tumors with low Tfam protein expression.

### Example 5

### Overexpression of Tfam Confers Apoptosis Resistance

Large-insert P1 artificial chromosomes (PACs) that contain the entire human TFAM gene and flanking regulatory sequences were cloned in our laboratory and injected to obtain PAC transgenic mice. We obtained different transgenic strains with a 1.5- and 5-fold increase of TFAM gene dosage and a corresponding increase of Tfam protein expression. We found a good correlation between increased TFAM gene dosage and increased levels of mtDNA and between increased TFAM gene dosage and increased levels of mtDNA transcripts. Animals with increased TFAM gene dosage were found to be more senstitive to radiation-induced in vivo apoptosis than their non-transgenic littermates. We established MEF cell lines from TFAM overexpressing animals, by using the same methods as described above. These cell lines had different TFAM gene dosage and there was a clear resistance to apoptosis induction by a variety of stimuli, including known apoptosis-inducing substances, chemotherapy agents and radiation, in cell lines with high TFAM gene dosage in comparison with cell lines with low TFAM gene dosage.

### Example 6

### An in Vitro Transcription System for Identifying Inhibitors and Activators of Human Mitochondrial Transcription

Human cells encode two proteins with sequence homology to the yeast mitochondrial transcription factor, Mtf1, also called mitochondrial RNA polymerase specificity factor, mitochondrial transcription factor B (sc-mtTFB). We have identified two human homologies of yeast Mtf1 and denote these proteins human TFB1M and TFB2M.

Recombinant human mitochondrial RNA polymerase (mtRNAP), TFAM, TFB1M, and TFB2M were expressed in a baculovirus system and purified to homogeneity. The activity of these proteins was studied in vitro by run off transcription assays. The template used was a human mtDNA fragment containing the light strand promoter (LSP) followed by a 200 base pairs long stretch of double-stranded DNA. Neither, mtRNAP, TFAM, TFB1M nor TFB2M did alone initiate transcription from the mtDNA promoter. Also mtRNAP together with either TFAM, TFB1M or TFB2M failed to initiate specific transcription. However, specific transcription initiation from LSP was obtained by combining either mtRNAP, TFAM and TFB1M or mtRNAP, TFAM and TFB2M. On the basis of these experiments we conclude that both TFB1M and TFB2M are functional homologues of the previously identified Saccharomyces cerevisiae protein Mtf1.

To investigate the promoter specificity of the transcription reaction, we added a double-stranded oligonucleotide containing only the LSP sequence in a 100-fold surplus to the template DNA. The promoter dependent transcription went down abut 15-fold in the presence of the LSP oligonucleotide. When the same experiment was repeated with an unrelated double stranded oligonucleotide of the same length, no effects on transcription could be observed.

We thus conclude that we have developed an in vitro transcription system with pure proteins that faithfully reproduces in vivo mtDNA transcription initiation. This system has allowed us to screen for low molecular compounds inhibiting or stimulating mitochondrial transcription.

### Example 7

### Antisense Inhibition of Nuclear Gene Products Regulating mtDNA Maintenance and Induces Apoptosis in Human and Mouse Fibroblasts

Antisense oligonucleotides were designed targeting the following human genes/mRNAs:

1. Mitochondrial DNA polymerase (catalytic subunit).

2. Mitochondrial RNA polymerase.

3. Mitochondrial transcription factor A (Tfam).

4. Mitochondrial transcription factor B (TFB1M, homologue to yeast Mtf1).

5. Mitochondrial transcription factor B (TFB2M, homologue to yeast Mtf1).

Two microliters of the 100 microM combinatorial oligonucleotide library (1.2×10¹⁴ independent oligonucleotide molecules) were allowed to hybridize to 5 microg of bead-immobilized mRNAs in vitro transcribed from human genes 1-5 (see above). As the randomized region of the library was set to be 18-mer, the input amount corresponds to an abundance of 100 molecules/18mer. In order not to disrupt the authentic secondary structure of the mRNA, the hybridization conditions of this experiment were set to very mild conditions, i.e., 37° C.-40° C. in 2×SST. After PCR amplification and cloning, accessible sequence tags were sequenced and based on these results we designed 5 antisense (and corresponding control mismatched) oligonucleotides for each of the four human genes listed above. These sequences were synthesized as phosphorothioates as well as phosphodiester/locked nucleic acid mix-mers.

Testing for antisense activities was carried out using human primary fibroblasts as well as other human cells. Typically, oligonucleotides (antisense and mismatched sequences) were added to the cell culture media in concentrations ranging from 0.01 to 10 microM for periods of 2-7 days with addition of fresh oligonucleotide at least once daily. Cells were then tested for changes in apoptosis markers, i.e. stainings, DNA laddering, Western blot analysis and FACS as described elsewhere in this document. Significant alterations is antisense but not mismatched sequence treated cultures with respect to these apoptosis markers were taken as evidence that targeted mRNA/gene/gene product (1-5 above) is essential for the expression of apoptosis.

### Example 8

### Identification of Putative Mouse and Human Homologs to the Yeast Mtf1 Protein

We used the profile-based PSI-BLAST method to search the NCBI sequence database, but found no mammalian homologues to S. cerevisiae Mtf1. However, by using the putative Schizosaccharomyces pombe Mtf1 homologue we identified a predicted protein, which we denoted TFB1M, with low, but significant sequence similarities to the yeast Mtf1 proteins (FIG. 5). The TFB1M sequence was, in turn, used for a BLASTP search of the NCBI sequence database and conserved homologues were identified in mouse, and Caenorhabditis elegans. Surprisingly, TFB1M also demonstrated sequence similarity to a second human and mouse protein, which we denoted TFB2M and Tfb2m, respectively, (FIG. 5b). TFB1M, Tfb1m, TFB2M, and Tfb2m all demonstrated highly significant homology to bacterial dimethyl transferases (FIG. 5b),

### Example 9

### Subcellular Localization and Tissue Expression Pattern for TFB1M and TFB2M

To experimentally verify that TFB1M and TFB2M were mitochondrial proteins, we performed confocal microscopy studies. Plasmids (pTfb1m-GFP and pTfb2m-GFP) encoding the complete amino acid sequence of the mouse Tfb1m and Tfb2m proteins, respectively, fused in frame to the green fluorescent protein (GFP) were constructed and used to transfect HeLa cells. A laser scanning confocal microscope was used to monitor the GFP reporter gene expression by observing excitation and emission at 488 nm and 400-440 nm, respectively. Mito-Tracker Red CMXRos (Molecular Probes) was added to living cells at a concentration of 25 nM for 20 minutes. Cells were observed with excitation light of 568 nm and emission light between 580-640 nm. Both Tfb1m-GFP and Tfb2m-GFP had a mitochondrial localization pattern indistinguishable from that of ornithine transcarbamylase (OTC)-GFP, a protein with known mitochondrial localization (FIG. 6A). Northern blot analyses showed that both the TFB1M and TFB2M genes were ubiquitously expressed (FIG. 6B), consistent with known expression patterns for other nucleus-encoded components of the mitochondrial transcription machinery.

### Example 10

### Both TFB1M and TFB2M Can Support in Vitro Transcription

For purification of His-mtRNAP/TFB1M or His-mtRNAP/TFP2M complexes, extracts from cells infected with His-tagged mtRNAP (5 pfu) together with either TFB1M (5 pfu) or TFB2M (5 pfu) were diluted 1:1 with buffer A (25 mM Tris-HCl (pH 8.0), 10% glycerol, protease inhibitors and 20 mM β-mercaptoethanol) containing 20 mM imidazole. Next 2 ml Ni²⁺-NTA matrix superflow (APBiotech) pre-equilibrated with buffer A, supplemented with 10 mM imidazole and 0.3. M NaCl, was added and incubated for 60 min at 4° C. with gentle rotation. The Ni²⁺-NTA matrix was collected by centrifugation (1500×g, 10 min, 4° C.), resuspended in buffer A (40 mM imidazole, 0.3 M NaCl), poured into a column and washed with 10 column volumes of the same buffer. The mtRNAP/TFB1M and mtRNAP/TFB2M complexes were eluted with buffer A (250 mM imidazole, 0.3 M NaCl). The peak fractions were dialyzed for 6 hrs against buffer B (25 mM Tris-HCl pH 8.0, 10% glycerol, 1 mM DTT, protease inhibitors, 0.5 mM EDTA) supplemented with 0.1M NaCl, frozen in liquid nitrogen, and stored at −80° C.

Expression of mtRNAP on its own was not successful, since most of the protein (>95%) proved insoluble. However, co-expression of TFB1M and mtRNAP or TFB2M and mtRNAP had a dramatic effect on the solubility of mtRNAP and only low levels (<5%) of insoluble polymerase was observed. The complexes purified after coexpression contained roughly equimolar amounts of TFB1M and mtRNAP or TFB2M and mtRNAP, respectively. For purification of isolated polymerase, His-mtRNAP was co-expressed with TFB2M and purified as described for His-mtRNAP/TFB2M, with the following modifications. The cellular extract was not diluted, but supplemented with 10 mM imidazole. Buffer A used for the Ni²⁺-NTA column, contained 1.0 M NaCl, which allowed for an effective separation of His-mtRNAP from TFB2M. His-mtRNAP was further purified on a 1 ml HiTrap heparin column (APBiotech) equilibrated in buffer B (0.1 M NaCl). After washing with three column volumes of buffer B (0.1 M NaCl), the proteins were eluted with a linear gradient (10 ml) of buffer B (0.1-1M), with His-mtRNAP protein eluting at 0.8M NaCl. The yield of His-mtRNAP protein from a 400 ml culture was approximately 2 mg. The purity of the protein was at least 95%, as estimated by SDS-polyacrylamide gel electrophoresis and Coomassie blue staining,

The His-tagged TFB2M protein was purified as His-mtRNAP, with the following modifications. The Sf9 cells were infected with 10 pfu of recombinant virus and the TFB2M protein eluted from the Hi-Trap Heparin column at 0.6 M NaCl. The His-TFB1M protein was purified as His-mtRNAP, with the following modifications. The Sf9 cells were infected with 10 pfu of recombinant virus. The HiTrap heparin column was eluted with a linear gradient (10 ml) of buffer B (0.5-1.5 M NaCl). The His-TFB1M eluted at about 1.3M NaCl and the yield of protein was approximately 6 mg from a 400 ml culture. The purity of the protein was at least 95%. The TFAM protein was purified as His-mtRNAP, with the following modifications. The Sf9 cells were infected with 10 pfu of recombinant virus. The dialyzed His-tagged TFAM from the Ni²⁺-NTA step was loaded on the MonoQ column equilibrated with buffer B (0.1M NaCl). The TFAM protein was in the flow through fractions. The yield of His-TFAM from a 400 ml culture was approximately 5 g with a purity of at least 95%. All proteins were frozen in aliquots on liquid nitrogen and stored at −80° C. FIG. 7 A is a SDS-PAGE gel stained with Coomassie blue depicting the different recombinant human proteins used for in vitro transcription reactions.

The in vitro transcription reactions were performed as follows:

DNA fragments corresponding to base pairs 1-741 (LSP/HSP), 1-477 (LSP) or 499-741 (HSP) of human mtDNA [Anderson et al., Nature 290:457 (1981)] were cloned into pUC18. The plasmid constructs were linearized and used to measure promoter specific transcription in a run off assay [Fisher et al., J. Biol. Chem. 260:11330 (1985)]. Individual reaction (25 μl) contained 85 fmol digested template, 10 mM Tris-HCl pH 8.0, 10 mM MgCl₂, 1 mM DTT, 100 μg/ml bovine serum albumin, 400 μM ATP, 150 μM CTP and GTP, 10 μM UTP, 0.2 μM α-³²P UTP (3000 Ci/mmol), 4 U Rnasin (APBiotech), and the indicated concentrations of proteins. After incubation at 32° C. for 30 min, the reactions were stopped by addition of 200 μl of stop buffer (10 mM Tris-HCl pH 8.0, 0.2 M NaCl, 1 mM EDTA, 0.1 mg/ml glycogen). Samples were treated with 0.5% SDS and 100 μg/ml proteinase K for 45 min at 42° C. and then precipitated by the addition of 0.6 ml ice cold ethanol. The pellets were dissolved in 10 μl of gel loading buffer (98% formamide, 10 mM EDTA pH 8.0, 0.025% xylene cyanol FF, 0.025% bromophenol blue), heated at 95° C. for 5 min, and analyzed on a 5% denaturing polyacrylamide gel in 1×TBE. The gels were fixed in 10% HAc, dried and exposed.

The experiments demonstrated that TFB1M and TFB2M were bona fide transcription factors and each of these factors could support promoter specific initiation of mitochondrial transcription in a recombinant in vitro system containing TFAM and mtRNAP/TFB1M or mtRNAP/TFB2M. In FIG. 7 B in vitro transcription is carried out in the presence of TFAM (2.5 pmol), mtRNAP/TFB1M (400 fmol) or mtRNAP/TFB2M (400 fmol). The transcriptional activation obtained with TFB2M is at least one order of magnitude greater than the activation obtained with TFB1M. Given the much higher activity and the of TFB2M, we focused our studies on characterization of TFB2M. To test if TFB2M could support transcription from both LSP and HSP we performed a transcription reaction using either of these two promoters or both theses two promoters on the same template (FIG. 7 C). The reactions contained TFAM (2.5 pmol) and mtRNAP/TFB2M (400 fmol). The experiments clearly demonstrates that TFB2M can support transcription from both LSP and HSP.

Given its dramatic effect on mtRNAP solubility, it was a formal possibility that TFB2M only was required for purification of mtRNAP without having a direct role in transcription. To address this question, we dissociated TFB2M and mtRNAP from each other at high salt concentration (1M NaCl) and further purified each factor to homogeneity. We used different combinations of pure mtRNAP (400 fmol), TFB2M (400 fmol) and TFAM (2.5 pmol) to support transcription from a LSP containing template and found that all three factors are needed for promoter specific initiation of transcription (FIG. 7D).

### Example 11

Using the in vitro transcription system described in example 10, we studied the ability of TFB2M to stimulate transcription by monitoring the effects of increasing amounts of TFB2M on LSP transcription at constant levels of TFAM and mtRNAP (FIG. 8A). Maximal transcription activity occurs at a 1:1 molar ratio of TFB2M and mtRNAP, whereas higher TFB2M concentrations did not stimulate transcription further. The in vitro transcription reaction mixtures contained 1.3 pmol of TFAM, 250 fmol of mtRNAP and 85 fmol of LSP-template. Increasing amounts of TFB2M were added as indicated. The molar ratio of TFB2M to mtRNAP and the relative levels of transcription are shown.

Given the absolute requirement of TFAM for in vitro transcription, we monitored the stimulatory effect of increasing amounts of TFAM on transcription initiation at the two promoters at constant levels of mtRNAP and TFB2M (FIG. 8B). The reaction mixture contained 400 fmol of mtRNAP, 400 fmol TFB2M, and 85 fmol of LSP/HSP-template. Increasing amounts of TFAM (0.025, 0.075, 0.25, 0.75, 2.5, 7.5, 15 and 22.5 pmol) were added. No transcription could be observed from either LSP or HSP in the absence of TFAM. LSP transcription was stimulated at low levels of TFAM and remained highly active at broad ranges of TFAM concentrations. In contrast, HSP transcription was only activated at a short interval of high TFAM concentration. A sharp decline of HSP and LSP transcription was observed when TFAM concentrations were increased further. These findings are in good agreement with previous studies using recombinant TFAM and a partially purified mtRNAP fraction [Parisi et al., Mol. Cell. Biol. 13:1951 (1993); Dairaghi et al., Bba-Mol Basis Dis 1271:127 (1995)]. Our results show that no additional factors besides TFAM, TFB2M and mtRNAP are required for establishing these promoter-specific transcription patterns and support the hypothesis by Clayton and coworkers suggesting that mitochondrial TFAM levels may differentially regulate HSP and LSP activation.

1. A method for inducing apoptosis of a living mammalian cell, comprising the steps of:
a) providing a substance capable of impairing mammalian mitochondrial DNA gene expression by affecting the expression of nuclear genes regulating mitochondrial DNA replication, mitochondrial DNA maintenance and stability, mitochondrial DNA transcription, the processing and stability of mitochondrial transcripts, mitochondrial protein translation or the stability of mitochondrially encoded proteins; and
b) administering said substance to said living mammalian cell in such an amount that apoptosis is induced.

2. A method according to claim 1, characterised in that said substance capable of impairing mammalian mitochondrial DNA gene expression comprises one or more antisense nucleic acid molecules.

3. A method according to claim 2, characterised in that said antisense nucleic acid molecule is complementary and/or specifically binding (targeting) a nuclear gene regulating mitochondrial DNA replication, mitochondrial DNA maintenance and stability, mitochondrial DNA transcription, the processing and stability of mitochondrial transcripts, mitochondrial protein translation or the stability of mitochondrially encoded proteins.

4. A method according to claim 2, characterised in that said antisense nucleic acid molecule is complementary and/or specifically binding (targeting) a nucleic acid molecule encoding a mitochondrial transcription factor.

5. A method according to claim 2, characterised in that said antisense nucleic acid molecule is complementary and/or specifically binding (targeting) a nucleic acid molecule encoding mitochondrial RNA polymerase (SEQ. ID. NO. 2).

6. A method according to claim 2, characterised in that said antisense nucleic acid molecule is complementary and/or specifically binding (targeting) a nucleic acid molecule encoding mitochondrial transcription factor A (TFAM) (SEQ. ID. NO. 4).

7. A method according to claim 2, characterised in that said antisense nucleic acid molecule is complementary and/or specifically binding (targeting) a nucleic acid molecule encoding the catalytic or accessory subunit of mitochondrial DNA polymerase (SEQ. ID. NO. 26, SEQ. ID. NO. 28).

8. A method according to claim 2, characterised in that said antisense nucleic acid molecule is complementary and/or specifically binding (targeting) a nucleic acid molecule encoding the mitochondrial transcription factor B (TFB1M or TFB2M) (SEQ. ID. NO. 6, SEQ. ID. NO. 8).

9. A method according to claim 2, characterised in that said antisense nucleic acid molecule is complementary and/or specifically binding (targeting) a nucleic acid molecule encoding Homo sapiens ribonuclease P and RNAse MRP subunits (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24).

10. An antisense nucleic acid molecule complementary and/or specifically binding (targeting) a nucleic acid molecule encoding mitochondrial RNA polymerase (SEQ. ID. NO. 2), mitochondrial transcription factor A (TFAM) (SEQ. ID. NO. 4), mitochondrial transcription factor B (TFB1M or TFB2M) (SEQ. ID. NO. 6, SEQ. ID. NO. 8), Homo sapiens ribonuclease P and RNAse MRP subunits (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), or mitochondrial DNA polymerase (SEQ. ID. NO. 26, SEQ. ID. NO. 28).

11. An antisense nucleic acid molecule complementary and/or specifically binding (targeting) a nuclear gene regulating mitochondrial DNA replication, mitochondrial DNA maintenance and stability, mitochondrial DNA transcription, the processing and stability of mitochondrial transcripts, mitochondrial protein translation or the stability of mitochondrially encoded proteins, for its medical use.

12. An antisense nucleic acid molecule complementary and/or specifically binding (targeting) a nucleic acid molecule encoding a mitochondrial transcription factor, for its medical use.

13. An antisense nucleic acid molecule complementary and/or specifically binding (targeting) a nucleic acid molecule encoding mitochondrial RNA polymerase (SEQ. ID. NO. 2), mitochondrial transcription factor A (TFAM) (SEQ. ID. NO. 4), mitochondrial transcription factor B (TFB1M or TFB2M) (SEQ. ID. NO. 6, SEQ. ID. NO. 8), Homo sapiens ribonuclease P and RNAse MRP subunits (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24), or mitochondrial DNA polymerase (SEQ. ID. NO. 26, SEQ. ID. NO. 28), for its medical use.

14. Use of one or more antisense nucleic acid molecules according to any one of claims 11 to 13 for preparing a pharmaceutical composition for treating cancer, lymphoproliferative syndromes, autoimmune diseases, sarcomas, meningeomas, basal cell carcinomas, benign tumors, psoriasis or prostatic hyperplasia.

15. A pharmaceutical composition for inducing apoptosis of a mammalian cell, comprising one or more antisense nucleic acid molecules according to any one of claims 11 to 13, together with a pharmaceutically acceptable carrier, excipient or diluent.

16. A method for in vitro identifying a substance capable of impairing mammalian mitochondrial DNA gene expression, thereby being capable of inducing apoptosis of a living mammalian cell, said method comprising the steps of:
a) providing a substance suspected of impairing mammalian mitochondrial DNA gene expression by affecting the expression of nuclear genes regulating mitochondrial DNA replication, mitochondrial DNA maintenance and stability, mitochondrial DNA transcription, the processing and stability of mitochondrial transcripts, mitochondrial protein translation or the stability of mitochondrially encoded proteins;
b) contacting the substance in step a) with a compound chosen from the group of
i. mitochondrial RNA polymerase (SEQ. ID. NO. 1) or the corresponding DNA/RNA sequence (SEQ. ID. NO. 2);
ii. mitochondrial transcription factor A (TFAM) (SEQ. ID. NO. 3)) or the corresponding DNA/RNA sequence (SEQ. ID. NO. 4);
iii. mitochondrial transcription factor B (TFB1M or TFB2M) (SEQ. ID. NO. 5, SEQ. ID. NO. 7)) or the corresponding DNA/RNA sequence (SEQ. ID. NO. 6, SEQ. ID. NO. 8);
iv. Homo sapiens ribonuclease P and RNAse MRP subunits (SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23)) or the corresponding DNA/RNA sequence (SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24);
v. the catalytic or accessory subunit of mitochondrial DNA polymerase (SEQ. ID. NO. 25, SEQ. ID. NO. 27)) or the corresponding DNA/RNA sequence (SEQ. ID. NO. 26, SEQ. ID. NO. 28); and
vi. fragments of the above compounds comprising at least 15 consecutive amino acids or at least 45 consecutive nucleotides; and
c) determining whether the substance in step a) binds to the compound of step b), thereby impairing mammalian mitochondrial DNA gene expression.

17. A method according to claim 16, characterised in that the compound in step b) is an enzyme chosen from mitochondrial RNA polymerase (SEQ. ID. NO. 1), TFAM (SEQ. ID. NO. 3), TFB1M or TFB2M (SEQ. ID. NO. 5, SEQ. ID. NO. 7), Homo sapiens ribonuclease P and RNAse MRP subunits (SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23), and mitochondrial DNA polymerase (SEQ. ID. NO. 25, SEQ. ID. NO. 27).

18. A method according to claim 17, characterised in that it is determined whether the substance in step a) upon contact affects the enzymatic activity of the enzyme in step b).

19. Use of a substance identified by the method of claims 16-18 for preparing a pharmaceutical composition for treating cancer, lymphoproliferative syndromes, autoimmune diseases, sarcomas, meningeomas, basal cell carcinomas, benign tumours, psoriasis, or prostatic hyperplasia, diabetes mellitus, heart failure, neurodegeneration, obesity or hormonal disturbances.
